(19) United States
(12) Patent Application Publication
(10) Pub. No.: US 2012/0258127 A1
(43) Pub. Date: Oct. 11, 2012

(54) VACCINE PEPTIDES

(76) Inventor: Simon Paul GRAHAM, Addlestone (GB)

(21) Appl. No.: 13/228,870

(22) Filed: Sep. 9, 2011

Related U.S. Application Data
(60) Provisional application No. 61/381,614, filed on Sep. 10, 2010.

(51) Int. Cl. C07K 7/08; G01N 21/64; A61P 31/14; G01N 33/566; C07H 21/04; A61K 39/12

(52) U.S. Cl. 424/186.1; 530/326; 536/23.72; 435/7.92; 435/7.1

(57) ABSTRACT

There is provided a polypeptide having an amino acid sequence selected from SEQ ID NOs:1, 2 and 3 or a functional fragment thereof, or comprising an amino acid sequence at least 65% identical to any one of SEQ ID NOs:1, 2 or 3. There is also provided a method of rapidly vaccinating an animal against infection by a pestivirus comprising administering to the animal an effective amount of a polypeptide comprising an E2 epitope polypeptide and/or an NS3 epitope polypeptide; the E2 epitope polypeptide may be SEQ ID NO:1 and the NS3 epitope polypeptide may be SEQ ID NO:2 or 3, or functional fragments thereof.

### CROSS-REFERENCE TO RELATED PATENT APPLICATIONS

This application claims priority to U.S. Provisional Patent Application No. 61/381,614, filed on Sep. 10, 2010, the disclosure of which is incorporated in its entirety.

### FIELD OF INVENTION

The invention relates to small polypeptides useful as vaccines to provide rapid protection to animals against infection by a pestivirus, within a few days of vaccination of the animal.

### BACKGROUND

Emergency vaccination plays a vital role in combating epizootic/epidemic diseases or biological warfare/terrorist attacks; critical to this intervention is the ability of the vaccines to induce rapid onset protection. Many inactivated and sub-unit vaccines require the administration of several injections over time to ensure high levels of protection, thus reducing their efficacy. On the other hand, live attenuated vaccines are often more efficacious and may confer rapid protection due to their ability to trigger innate pathways, leading to the induction of protective cell mediated immune responses. However, many live attenuated vaccines carry genuine concerns over their safety, due to the potential of reversion to virulence and recombination with pathogens in the field.

Classical swine fever (CSF) is a devastating disease that poses one of the greatest risks to the swine industry worldwide, both from an economic and sanitary perspective. CSF is caused by the classical swine fever virus (CSFV), a highly contagious, small, enveloped, single-stranded RNA virus that belongs to the family Flaviviridae, which also includes Bovine Viral Diarrhoea Virus (BVDV) and Border Disease Virus (BDV). CSF has, since 1990, principally been controlled in the EU through a ‘stamping-out’ slaughter policy, but the presence of a CSFV reservoir in European wild-boar populations, together with increasing public opposition against stamping-out practices, has now led to an increased likelihood that vaccination may be deployed as a last resort component of a control strategy (van Oirschot (2003) Developmental Biology (Base1) 114: 259-267). Existing live attenuated CSFV vaccines, such as those based on lapinised or culture attenuated C-strain viruses, provide a rapid onset of complete protection, but pose problems in discriminating infected amongst vaccinated animals, whereas questions remain about the efficacy of marker sub-unit vaccines for use under emergency outbreak conditions (van Oirschot (2003) Vet. Microbiol. 96: 367-384).

The immunological mechanisms that underlie the rapid protection afforded by live, attenuated C-strain vaccines are not well defined. However, protection may precede the appearance of neutralising antibody but not IFN-γ secreting cells in peripheral blood (Suradhat & Damrongwatanapokin (2003) Vet. Microbiol. 92: 187-194), suggesting that cellular immunity may be responsible.

### SUMMARY OF THE INVENTION

The present invention provides isolated polypeptides comprising an E2 epitope or an NS3 epitope. In one embodiment of the invention, the E2 epitope is a B-cell epitope from the E2 glycoprotein and the NS3 epitope is a T-cell epitope from the NS3 protein, both classical swine fever virus (CSFV), Bovine Viral Diarrhoea Virus (BVDV) and/or Border Disease Virus (BDV).

In one embodiment, the isolated polypeptide may comprise the amino acid sequence of SEQ ID NO: 1, 2, or 3. In another embodiment, the polypeptide may comprise an E2 epitope which is the amino acid sequence of SEQ ID NO: 1. In another embodiment, the polypeptide may comprise a NS3 epitope which is the amino acid sequence of SEQ ID NO: 2 or 3. In a further embodiment, the polypeptide may comprise an E2 epitope which consists of the amino acid sequence of SEQ ID NO: 1. In a further embodiment, the polypeptide may comprise a NS3 epitope which consists of the amino acid sequence of SEQ ID NO: 2 or 3. In a further embodiment, the polypeptide may comprise a functional fragment of the amino acid sequence of SEQ ID NO: 1, 2, or 3. In another embodiment, the functional fragment may comprise at least about 8, 9, 10, 11, 12, 13, 14 or 15 contiguous amino acids from the amino sequence SEQ ID NO:1, 2 or 3. Typical functional properties of functional fragments include, but are not limited to ability to, eliciting an immune responses, increasing IFN-γ levels, or elicit an immune response, either a humoral immune response and/or a cellular immune response, similar to that elicited by the full-length polypeptide.

In one embodiment, the isolated polypeptide may comprise a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3. In particular embodiment, the polypeptide may comprise a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1. In still another embodiment, the polypeptide may comprise a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 2. In another embodiment, the polypeptide may comprise a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 3. The variant polypeptides of the amino acid sequence of SEQ ID NO: 1, 2, or 3 may have functional properties including but are not limited to eliciting an immune responses (e.g., a humoral immune response and/or a cellular immune response), increasing IFN-γ levels, or eliciting an immune response, either a humoral immune response and/or a cellular immune response, similar to that elicited by a polypeptide comprising the amino acid sequence of SEQ ID NO: 1, 2, or 3.

In a further embodiment, the isolated polypeptide may comprise a functional fragment of a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3. The functional fragments of variant polypeptides of the amino acid sequence of SEQ ID NO: 1, 2, or 3 may have functional properties including but are not limited to ability to elicit an immune responses such as a humoral immune response and/or a cellular immune response, increase IFN-γ levels, or elicit an immune response, either a humoral immune response and/or a cellular immune response, similar to a polypeptide comprising the amino acid sequence of SEQ ID NO: 1, 2, or 3.

The invention further provides fusion proteins comprising a polypeptide comprising the amino acid sequence of SEQ ID NO: 1, 2, or 3 and a heterologous peptide, which may comprise a histidine tag. In another embodiment, the fusion protein comprises a functional fragment of the amino acid sequences of SEQ ID NO: 1, 2, or 3 and a heterologous peptide. In a further embodiment, the fusion protein may comprise a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3 and a heterologous peptide. In a further embodiment, the fusion protein may comprise a functional fragment of a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3 and a heterologous peptide. In one embodiment, the heterologous peptide is a heptahistidine tag.

The invention also provides an isolated polynucleotide encoding at least one polypeptide comprising the amino acid sequence of SEQ ID NO: 1, 2, or 3. In a particular embodiment, the isolated polynucleotide the amino acid sequence of SEQ ID NO: 1. In a another embodiment, the isolated polynucleotide encodes the amino acid sequence of SEQ ID NO: 2 or 3. In a further embodiment, the isolated polynucleotide encodes a polypeptide may comprise a functional fragment of the amino acid sequence of SEQ ID NO: 1, 2, or 3. In a further embodiment, the isolated polynucleotide encodes a polypeptide may comprise a functional fragment of the amino acid sequence of SEQ ID NO: 1, 2, or 3. The invention also provides for an expression vector comprising a polynucleotide encoding at least one polypeptide comprising the amino acid sequence of SEQ ID NO: 1, 2, or 3, or a functional fragment thereof. In another embodiment, the invention provides a host cell comprising an expression vector comprising a polynucleotide encoding at least one polypeptide comprising the amino acid sequence of SEQ ID NO: 1, 2, or 3, or a functional fragment thereof.

In one embodiment, the invention provides a pharmaceutical composition comprising a polypeptide comprising the amino acid sequence of SEQ ID NO: 1, 2, or 3. In another embodiment, the composition may comprise a polypeptide that may comprise an E2 epitope which is the amino acid sequence of SEQ ID NO: 1. In another embodiment, the composition may comprise a polypeptide may comprise a NS3 epitope which is the amino acid sequence of SEQ ID NO: 2 or 3. In a further embodiment, the composition may comprise a polypeptide comprising an E2 epitope which consists of the amino acid sequence of SEQ ID NO: 1. In a further embodiment, the composition may comprise a polypeptide comprising a NS3 epitope which consists of the amino acid sequence of SEQ ID NO: 2 or 3. In a further embodiment, the composition may comprise a polypeptide may comprise a functional fragment of the amino acid sequence of SEQ ID NO: 1, 2, or 3. In another embodiment, the composition may comprise a polypeptide that comprises a functional fragment with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3.

In one embodiment, the invention provides a composition comprising a polynucleotide encoding a polypeptide comprising the amino acid sequence of SEQ ID NO: 1, 2, or 3. In another embodiment, the composition may comprise a polynucleotide encoding a polypeptide that comprises an E2 epitope. In another embodiment, the composition may comprise a polynucleotide encoding a polypeptide that comprises a NS3 epitope. In a further embodiment, the composition may comprise a polynucleotide encoding a polypeptide that comprises an E2 epitope which consists of the amino acid sequence of SEQ ID NO: 1. In a further embodiment, the composition may comprise a polynucleotide encoding a polypeptide that comprises a NS3 epitope which consists of the amino acid sequence of SEQ ID NO: 2 or 3. In a further embodiment, the composition may comprise a polynucleotide encoding a polypeptide that comprises a functional fragment of the amino acid sequence of SEQ ID NO: 1, 2, or 3. In a further embodiment, the composition may comprise a polynucleotide encoding a polypeptide that comprises a functional fragment of the amino acid sequence of SEQ ID NO: 1, 2, or 3. In another embodiment, the composition may comprise a polynucleotide encoding a polypeptide that comprises a functional fragment with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3.

In another embodiment, the composition may comprise at least one excipient, carrier, or adjuvant. In one embodiment, the composition may be an antigenic composition. In an alternate embodiment, the composition may be an immunogenic composition. In a still another embodiment, the composition may be a pharmaceutical composition. In a further embodiment, the composition is comprises a pharmaceutical carrier. In a further embodiment, the composition may elicit an immune response. In another embodiment, the immune response may be a protective immune response. In one embodiment, the composition may elicit a humoral immune response, wherein said humoral immune response may be specific for the E2 epitope. In one embodiment, the composition may elicit a cellular immune response, wherein said cellular immune response may be specific for the NS3 epitope.

In another embodiment, the invention provides a vaccine preparation comprising a polypeptide comprising the amino acid sequence of SEQ ID NO: 1, 2, or 3. In another embodiment, the vaccine preparation comprises a polypeptide consisting of the amino acid sequence of SEQ ID NO: 1, 2, or 3. In a further embodiment, the vaccine preparation comprises a polypeptide that comprises a functional fragment of the amino acid sequence of SEQ ID NO: 1, 2, or 3. In a further embodiment, the vaccine preparation comprises a polypeptide that comprises a functional fragment of the amino acid sequence of SEQ ID NO: 1, 2, or 3. In another embodiment, the polypeptide that comprises a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3. In another embodiment, the vaccine preparation comprises a polypeptide comprising an E2 epitope. In a further embodiment, the vaccine preparation comprises a polypeptide comprising an E2 epitope that comprises a functional fragment the amino acid sequence of SEQ ID NO: 1. In another embodiment, the vaccine preparation comprises a polypeptide comprising a NS3 epitope. In a further embodiment, the vaccine preparation comprises a polypeptide comprising a NS3 epitope which consists of a functional fragment the amino acid sequence of SEQ ID NO: 2 or 3. In a further embodiment, the vaccine preparation elicits an immune response. In another embodiment, the immune response may be a protective immune response. In one embodiment, the vaccine preparation elicits a humoral immune response, where said humoral immune response may be specific for the E2 epitope. In one embodiment, the vaccine preparation elicits a cellular immune response, where said cellular immune response may be specific for the NS3 epitope.

In another embodiment, the invention provides a vaccine preparation comprising a polynucleotide encoding a polypeptide comprising the amino acid sequence of SEQ ID NO: 1, 2, or 3. In another embodiment, the vaccine preparation comprises a polynucleotide encoding a polypeptide consisting of the amino acid sequence of SEQ ID NO: 1, 2, or 3. In a further embodiment, the vaccine preparation comprises a polynucleotide encoding a polypeptide which comprises a functional fragment of the amino acid sequence of SEQ ID NO: 1, 2, or 3. In a further embodiment, the vaccine preparation comprises a polynucleotide encoding a polypeptide may comprise a functional fragment of the amino acid sequence of SEQ ID NO: 1, 2, or 3. In another embodiment, vaccine preparation comprises a polynucleotide encoding a polypeptide with at least 65%, 70%, 75%, 80%, 85%, 90%, 91%, 92%, 93%, 94%, 95%, 96%, 97%, 98%, or 99% sequence homology with the amino acid sequence of SEQ ID NO: 1, 2, or 3. In another embodiment, the vaccine preparation comprises a polynucleotide encoding a polypeptide comprising an E2 epitope which consists of the amino acid sequence of SEQ ID NO: 1. In a further embodiment, the vaccine preparation comprises a polynucleotide encoding a polypeptide comprising an E2 epitope which consists of a functional fragment the amino acid sequence of SEQ ID NO: 1. In another embodiment, the vaccine preparation comprises a polynucleotide encoding a polypeptide comprising a NS3 epitope which consists of the amino acid sequence of SEQ ID NO: 2 or 3. In a further embodiment, the vaccine preparation comprises a polynucleotide encoding a polypeptide comprising a NS3 epitope which consists of a functional fragment the amino acid sequence of SEQ ID NO: 2 or 3. In a further embodiment, the vaccine preparation elicits an immune response. In another embodiment, the immune response may be a protective immune response. In one embodiment, the vaccine preparation elicits a humoral immune response, where said humoral immune response may be specific for the E2 epitope. In one embodiment, the vaccine preparation elicits a cellular immune response, where said cellular immune response may be specific for the NS3 epitope.

In one embodiment, the invention provides for a method of rapidly vaccinating an animal against infection by a pestivirus comprising administering to the animal an effective amount of a polypeptide comprising an E2 epitope polypeptide and/or an NS3 epitope polypeptide. In another embodiment, the E2 epitope polypeptide comprises SEQ ID NO:1 and/or a functional fragment thereof and/or an amino acid sequence at least 65% identical to SEQ ID NO:1 and wherein the NS3 epitope polypeptide comprises SEQ ID NO:2 and/or 3 and/or a functional fragment thereof and/or an amino acid sequence at least 65% identical to any one of SEQ ID NOs:2 or 3. In one embodiment, the vaccine preparation comprises a polynucleotide comprising a polypeptide comprising the amino acid sequence of SEQ ID NO: 1, 2, or 3, or a functional fragment thereof. In a further embodiment, the animal may be protected from infection from at least 1 day after administration of the polypeptide. In a further embodiment, the animal may be protected from infection from at least 3 days after administration of the polypeptide. In a further embodiment, the animal may be protected from infection from at least 5 days after administration of the polypeptide. In a further embodiment, the pestivirus is selected from Classical Swine Fever Virus (CSFV) or Bovine Diarrhoea Virus (BVDV) or Border Disease Virus (BDV). In another embodiment, the animal may be a cetartiodactyla mammal. In a further embodiment, the animal may be a mammal. In another embodiment, the animal may be a pig, cow, or sheep. In a further embodiment, the composition may elicit an immune response. In another embodiment, the immune response may be a protective immune response. In one embodiment, the composition may elicit a humoral immune response, where said humoral immune response may be specific for the E2 epitope. In one embodiment, the composition may elicit a cellular immune response, where said cellular immune response may be specific for the NS3 epitope.

In one embodiment, the invention provides for a method of eliciting an immune response specific for a pestivirus in an animal comprising administering to the animal a composition comprising an effective amount of a polypeptide comprising an E2 epitope polypeptide and/or an NS3 epitope polypeptide. In one mode, the E2 epitope polypeptide comprises SEQ ID NO: 1, or a functional fragment thereof. In another mode, the NS3 epitope polypeptide comprises SEQ ID NO: 2 or 3, or a functional fragment thereof. In one embodiment, the vaccine preparation comprises a polynucleotide encoding a polypeptide comprising the amino acid sequence of SEQ ID NO: 1, 2, or 3, or a functional fragment thereof. In a further embodiment, the animal may be protected from infection from at least 1 day after administration of the polypeptide. In a further embodiment, the animal may be protected from infection from at least 3 days after administration of the polypeptide. In a further embodiment, the animal may be protected from infection from at least 5 days after administration of the polypeptide. In a further embodiment, the pestivirus is selected from Classical Swine Fever Virus (CSFV) or Bovine Diarrhoea Virus (BVDV) or Border Disease Virus (BDV). In another embodiment, the animal may be a cetartiodactyla mammal. In a further embodiment, the animal may be a mammal. In another embodiment, the animal may be a pig, cow, or sheep. In another embodiment, the composition may comprise at least one excipient, carrier, or adjuvant. In one embodiment, the composition may be an antigenic composition. In one embodiment, the composition may be an immunogenic composition. In a further embodiment, the composition may be a pharmaceutical composition. In another embodiment, the composition is comprises a pharmaceutical carrier. In a further embodiment, the composition may elicit an immune response. In another embodiment, the immune response may be a protective immune response. In one embodiment, the composition may elicit a humoral immune response, where said humoral immune response may be specific for the E2 epitope. In one embodiment, the composition may elicit a cellular immune response, where said cellular immune response may be specific for the NS3 epitope.

The invention also provides for a method of determining whether an animal has been exposed to infection by a pestivirus comprising contacting a sample of cells with a polypeptide comprising an E2 epitope polypeptide and/or an NS3 epitope polypeptide and determining whether levels of IFN-γ are increased in said cells after the contacting, an increase in IFN-γ levels being indicative of exposure of the animal to the virus. In one embodiment, the E2 epitope polypeptide comprises SEQ ID NO:1 and/or a functional fragment thereof and/or an amino acid sequence at least 65% identical to SEQ ID NO:1 and wherein the NS3 epitope polypeptide comprises SEQ ID NO:2 and/or 3 and/or a functional fragment thereof and/or an amino acid sequence at least 65% identical to any one of SEQ ID NOs:2 or 3. In a further embodiment, the pestivirus is selected from Classical Swine Fever Virus (CSFV) or Bovine Diarrhoea Virus (BVDV) or Border Disease Virus (BDV). In another embodiment, the animal may be a cetartiodactyla mammal. In a further embodiment, the animal may be a mammal. In another embodiment, the animal may be a pig, cow, or sheep.

### BRIEF DESCRIPTION OF THE FIGURES

FIG. 1 depicts characterization of serum neutralising antibody and type I IFN responses following vaccination with a live attenuated C-strain CSFV and challenge with genotypically diverse CSFV isolates, (A, C) UK2000 7.1 or (B, D) CBR93 CSFV isolates.

FIG. 2 depicts characterization of virus specific IFN-γ responses following vaccination with a live attenuated C-strain CSFV and challenge with genotypically diverse CSFV isolates, (A, B) UK2000/7.1 or (C, D) CBR93 CSFV isolates.

FIG. 3 depicts association of vaccine-primed virus specific IFN-γ responses and viraemia, following challenge with CSFV; pigs were vaccinated on days-5 (A), -3 (B) or -1 (C, animals that experienced mild signs followed by recovery and D, animals that developed severe signs necessitating euthanasia) and were then challenged (together with (E), unvaccinated pigs) with UK2000 7.1 or CBR93 CSFV.

FIG. 4 depicts analyses of the cross-reactivity and specificity of virus specific T cell IFN-γ responses.

FIG. 5 depicts results of phenotyping of virus-specific IFN-γ secreting T cells by flow cytometry.

FIG. 6 depicts effects of porcine IFN-γ on CSFV infection in vitro, with representative results of one of two independent experiments shown.

### SUMMARY OF INVENTION

According to a first aspect of the invention, there is provided a polypeptide having an amino acid sequence selected from SEQ ID NOs:1, 2 and 3 or a functional fragment thereof, or having an amino acid sequence at least 65% identical to any one of SEQ ID NOs:1, 2 or 3. The polypeptide is preferably isolated. The polypeptides are useful as vaccines to provide rapid protection to an animal against infection by a pestivirus, with the animal being protected from infection from a short time after the vaccine has been administered, for example, between 1-10 days, for example, from about 1, 2, 3, 4, 5 or about 6 days from administration.

The polypeptide may consist of an amino acid sequence selected from SEQ ID NO:1, 2 or 3 or a functional fragment of any of these. The polypeptide may also comprise more than one of the sequences or a combination of sequences, as well as other polypeptide sequences. Therefore, in a related aspect, the polypeptide may be a fusion protein comprising a polypeptide according to the invention and one or more other polypeptide sequences.

According to a second aspect of the invention, there is provided a polynucleotide encoding at least one polypeptide according to the first aspect of the invention. Such polynucleotides can be used in a polynucleotide vaccine, so that the polypeptide is expressed within a cell of an animal to which the vaccine has been administered, the polypeptide then being able to act to provide protection to the animal against infection by a pestivirus. The polynucleotide may form part of an expression vector comprising other components necessary for DNA transcription and subsequent protein expression to occur and the skilled person is readily able to determine such components.

Therefore, a vaccine preparation according to a third aspect of the invention may comprise a polypeptide according to the first aspect of the invention or a polynucleotide according to the second aspect of the invention.

The vaccine compositions of the invention may be subunit vaccines comprising the immunologically active peptides or a lipopeptide derived therefrom.

For subunit vaccines, the polypeptide of the invention may conveniently be formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the vaccine composition are adjusted according to routine skills.

Optionally, the vaccine formulation may include a carrier. Commonly used carrier molecules are bovine serum albumin (BSA), keyhole limpet hemocyanin (KLH), ovalbumin, mouse serum albumin, rabbit serum albumin and the like. Synthetic carriers may be used and are readily available. Means for conjugating peptides to carrier proteins are well known in the art and include glutaraldehyde, m-maleimidobenzoyl-N-hydroxysuccinimide ester, carbodiimide and bis-biazotized benzidine.

In certain situations, it may also be desirable to formulate the polypeptide with an adjuvant to enhance the immune response. Such adjuvants include all acceptable immunostimulatory compounds such as, for example, a cytokine, toxin, or synthetic composition.

In addition to adjuvants, it may be desirable to co-administer biologic response modifiers (BRM) with the peptide or variant or derivative to down regulate suppressor T cell activity.

Possible vehicles for administration of the vaccine formulation include liposomes. Liposomes are microscopic vesicles that consist of one or more lipid bilayers surrounding aqueous compartments. Liposomes are similar in composition to cellular membranes and, as a result, liposomes generally can be administered safely and are biodegradable. Techniques for preparation of liposomes and the formulation (e.g., encapsulation) of various molecules, including peptides and oligonucleotides, with liposomes are well known.

Depending on the method of preparation, liposomes may be unilamellar or multilamellar, and can vary in size with diameters ranging from 0.02 μm to greater than 10 μm. Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents. In the present context, the polypeptide according to the invention can be localized on the surface of the liposome, to facilitate antigen presentation without disruption of the liposome or endocytosis. Irrespective of the mechanism or delivery, however, the result is the intracellular disposition of the associated polypeptide.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MIN), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). Techniques for preparing these liposomes are well known in the art.

Other forms of delivery particle, for example, microspheres and the like, also are contemplated for delivery of the peptide epitopes or polyepitopes.

Alternatively, nucleic acid-based vaccines may be produced that comprise nucleic acid, such as, for example, DNA or RNA, encoding the immunologically active peptide epitope or polyepitope and cloned into a suitable vector (e.g., vaccinia, canarypox, adenovirus, or other eukaryotic virus vector).

Alternatively, the polypeptide may be administered in the form of a cellular vaccine via the administration of autologous or allogeneic APCs or dendritic cells that have been treated in vitro so as to present the peptide on their surface.

According to a fourth aspect of the invention, there is provided a method of rapidly vaccinating an animal against infection by a pestivirus comprising administering to the animal an effective amount of a polypeptide comprising an E2 epitope polypeptide and/or an NS3 epitope polypeptide. The polypeptide may be isolated in that it is prepared in the absence of other peptide components such as virus outer membrane proteins, for example. The fourth aspect may comprise administering to the animal an effective amount of a vaccine preparation according to the third aspect of the invention.

The term “epitope polypeptide”, as used throughout this specification, indicates a peptide which contains one or more (or all) T-cell epitopes of the relevant protein. Therefore, an E2 epitope polypeptide is a polypeptide comprising the E2 full length polypeptide or a fragment thereof which contains at least one E2 T-cell epitope and a NS3 epitope polypeptide is a polypeptide comprising the NS3 full length polypeptide or a fragment thereof which contains at least one NS3 T-cell epitope. The polyprotein of BVDV Oregon C24V strain, comprising E2 and NS3, has GenBank accession no. AF091605 (SEQ ID NO:4).

The term “epitope” refers to the amino acids (typically a group of around 8 or more amino acids) within a peptide sequence which are essential in the generation of an immune response and which can, therefore, be used as a vaccine or in a diagnostic test. The immune response may be an antibody mediated immune response or a non-antibody mediated immune response, for example, an immune response which can be detected by means of a cell-mediated immunity (CMI) assay such as an IFN-γ assay. Therefore, the epitope is one which is recognisable by a T cell, for example by binding of a T cell receptor to the epitope.

The E2 epitope polypeptide may comprise SEQ ID NO:1 and/or a functional fragment thereof and/or an amino acid sequence at least 65% identical to SEQ ID NO:1. The NS3 epitope polypeptide may comprise SEQ ID NO:2 and/or 3 and/or a functional fragment thereof and/or an amino acid sequence at least 65% identical to any one of SEQ ID NOs:2 or 3. The polypeptide may be administered directly or as the result of cellular expression following administration of a polynucleotide encoding the polypeptide. Therefore, the method may be conducted by administering a polypeptide, polynucleotide or vaccine preparation according to previous aspects of the invention.

Use of the method may result in the animal being rapidly protected from infection from at least 1 day after administration of the polypeptide, or at least about 3 days or at least about 5 days from infection. “Protection from infection” indicates that, if the animal is exposed to an infectious agent such as a pestivirus on the first, third or fifth day (for example, as appropriate) after administration of the vaccine, the animal does not go on to exhibit symptoms of the disease caused by the pestivirus (for example, Classical swine fever in the case of CSFV). In addition, the animal and/or cells from the animal may exhibit elevated IFN-γ levels after challenge with an infectious agent on the relevant day after administration of the vaccine occurred. These properties may be evident before antibodies against the polypeptide are detectable. The term “rapidly vaccinating” indicates that protection of the animal against infection has occurred within the short time periods (i.e., 1-10 days) after vaccination as described herein.

The pestivirus may be selected from Classical Swine Fever Virus (CSFV) or Bovine Diarrhoea Virus (BVDV) or Border Disease Virus (BDV). The animal may be a cetartiodactyla mammal such as a pig, cow or sheep.

According to a fifth aspect of the invention, there is provided a method of determining whether an animal has been exposed to a pestivirus or a component thereof, comprising contacting a sample of cells from the animal with a polypeptide comprising an E2 epitope polypeptide and/or an NS3 epitope polypeptide and determining whether levels of IFN-γ are increased in said cells after the contacting, an increase in IFN-γ levels being indicative of exposure of the animal to the virus or a component thereof. The increase may be determined by comparison of IFN-γ levels in an equivalent animal (i.e., an animal of the same species and, perhaps, breed) which is known to not have been exposed to the virus or component thereof, i.e., a negative control.

The E2 epitope polypeptide may comprise SEQ ID NO:1 and/or a functional fragment thereof and/or an amino acid sequence at least 65% identical to SEQ ID NO:1. The NS3 epitope polypeptide may comprise SEQ ID NO:2 and/or 3 and/or a functional fragment thereof and/or an amino acid sequence at least 65% identical to any one of SEQ ID NOs:2 or 3. Therefore, the method may be carried out by determining a change in expression of IFN-γ following contacting the cells with a polypeptide, polynucleotide or vaccine preparation according to earlier aspects of the invention. The pestivirus may be selected from Classical Swine Fever Virus (CSFV) or Bovine Diarrhoea Virus (BVDV) or Border Disease Virus (BDV). The animal may be a cetartiodactyla mammal such as a pig, cow or sheep.

The sample of cells may be obtained and isolated from an animal and contacted with the polypeptide (or polynucleotide or vaccine preparation) in vitro, for example as described elsewhere in the present application.

The present invention also encompasses polypeptides comprising variants of the epitope polypeptides and methods utilising these variant polypeptides. As used herein, a “variant” means a polypeptide in which the amino acid sequence differs from the base sequence from which it is derived in that one or more amino acids within the sequence are deleted or substituted with other amino acids. The variant is a functional variant, in that the functional characteristics of the polypeptide from which the variant is derived are maintained. For example, a similar immune response is elicited by exposure of an animal, or a sample from an animal, to the variant polypeptide. In particular, any amino acid substitutions, additions or deletions must not alter or significantly alter the tertiary structure of one or more epitopes contained within the polypeptide from which the variant is derived. The skilled person is readily able to determine appropriate functional variants and to determine the tertiary structure of an epitope and any alterations thereof, without the application of inventive skill.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | Ala, Val, Leu, Ile, Pro, Met, Phe, Trp |
| Uncharged polar: | Gly, Ser, Thr, Cys, Tyr, Asn, Gln |
| Acidic: | Asp, Glu |
| Basic: | Lys, Arg, His |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the peptide's conformation.

As mentioned above, non-conservative substitutions are possible provided that these do not disrupt the tertiary structure of an epitope within the polypeptide, for example, which do not interrupt the immunogenicity (for example, the antigenicity) of the peptide.

Broadly speaking, fewer non-conservative substitutions will be possible without altering the biological activity of the polypeptide. Suitably, variants may be at least about 65% identical, about 70% identical, for example at least about 75% identical, such as at least about 80%, 85%, 90%, 91%, 92%, 93%, 94%, 95%, 96%, 97%, 98% or about 99% identical to the base sequence.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. The percentage sequence identity may be determined using BLAST sequence alignment software, publicly available via http://blast.ncbi.nlm.nih.gov/Blast.cgi (accessible on 10 Sep. 2010), using default parameter settings.

A functional fragment of the polypeptide is a fragment wherein the functional characteristics of the polypeptide from which the fragment is derived are maintained, as described above. A functional fragment of any of SEQ ID NOs:1, 2 or 3 may comprise, for example, 8, 9, 10, 11, 12, 13, 14 or 15 contiguous amino acids from within the sequence SEQ ID NO:1, 2 or 3.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### EXAMPLES

The invention now being generally described, it will be more readily understood by reference to the following examples, which are included merely for purposes of illustration of certain aspects and embodiments of the present invention, and are not intended to limit the invention.

### Example 1

### Materials and Methods

Animals and Viruses.

Forty-four ‘high-health’ status Large White/Landrace cross male pigs, 8-10 weeks of age, were purchased from a local commercial source. Lyophilized, live attenuated Riemser C-strain CSFV (AC Reimser Schweinepestvakzine, Reimser Arzneimittel AG, Germany) was provided by the European Commission Vaccine Bank. For inoculation of pigs, the virus was reconstituted in vaccine diluent as described by the manufacturer. The CSFV isolates Eystrup and Alfort 187 (Genotype 1.1), Baker A and Brescia (Genotype 1.2), Guatemala (Genotype 1.3), UK2000/7.1 and Alfort Tthingen (Genotype 2.1), SP339106 (Genotype 2.2), Rostock (Genotype 2.3), Congenital Tremor (Genotype 3.1), CBR93 (Genotype 3.3), Kanagawa (Genotype 3.4) and BVDV Oregon C24V were obtained from the Mammalian Virus Investigation Unit (VLA-Weybridge, UK). Additional CSFV stocks were prepared following inoculation of sub-confluent porcine kidney cell (PK-15) monolayers and virus titres determined as previously described (Drew (2008) Classical swine fever (hog cholera) In: OIE Manual of Diagnostic Tests and Vaccines for Terrestrial Animals (mammals, birds and bees) Sixth Edition; ISBN 978-92-9044-718-4; Vol. 2. Chapter 2.8.3. p. 1092-1106).

Proteins and Peptides.

Recombinant CSFV Alfort Tübingen proteins (residue numbering refers to CSFV Alfort Tübingen polyprotein; Genbank accession number J04358.2); N^{pro} (AA 1-168), core (AA 169-250), NS2/3 protease (1435-1780), NS3 helicase domain (AA 1782-2272), NS4A (AA 2273-2336), NS4B (AA 2461-2683), NS5A (AA 2684-3180) and NS5B (AA 3181-3898, were expressed as N- or C-terminal fusions with heptahistidine tags in E. coli and applied to HisTrap columns (GE Life Science) using native buffer (buffer A: 500 mM NaCl, 50 mM Na H2PO4, pH 7.4) (Npro, Core, NS3, NS4A) or denaturing buffer (8M urea or 4M guanidine hydrochloride in buffer A) conditions (NS2, NS4B, NS5B). Proteins were eluted with gradients of 100-500 mM imidazole dissolved in the same buffers. Only proteins with purities of >90% were included in the experiments.

Synthetic overlapping peptides spanning the entire length of the polyprotein of BVDV, Oregon C24V (GenBank accession no. AF091605) (16mers offset by four residues), were obtained from Pepscan Systems B.V. (Lelystad, The Netherlands). Peptides were dissolved at a concentration of 5 mg/ml in 50% (v/v) DNA synthesis grade acetonitrile/water (Applied Biosystems, Warrington, UK), aliquoted and stored at −20° C. Peptides were combined to obtain pools representative of the 12 pestiviral proteins. However, because of their size, the pools corresponding to NS3 and NS5B were split in two, with peptides corresponding to the N-terminal half being pooled in pool I and C-terminal peptides in pool II.

Vaccination and Challenge with CSFV.

In each of two independent experiments, pigs were randomised into 4 groups, with groups 1-3 each consisting of 6 pigs and group 4 containing 4 pigs. Groups 1-3 were vaccinated intramuscularly with 100PD₅₀ (1 ml; <100 TCID₅₀) of the attenuated C-strain of CSFV on days-5, -3 and -1 respectively. On day 0, all 4 groups were inoculated intra-nasally with 10^{5.3} TCID₅₀ (2 ml divided equally between each nostril) of the UK2000/7.1 CSFV isolate or the CBR93 CSFV isolate. For each experiment, experimental groups were housed in separate pens. All work was carried out in accordance with UK legislation pertaining to care and use of animals under experimentation.

Vaccination with Epitope Polypeptides.

Pigs were vaccinated intramuscularly with 100 μg of one of the peptides having sequence SEQ ID NOs:1, 2 or 3, diluted in PBS and prepared in commercial adjuvant Emulsigen™-D (MVP technologies, Omaha, USA). Vaccination was at days-5, -3 and -1, with a control group of animals being unvaccinated. On day 0, all animals were inoculated intra-nasally with 10^{5.3} TCID₅₀ (2 ml divided equally between each nostril) of a CSFV isolate. For each experiment, experimental groups were housed in separate pens. All work was carried out in accordance with UK legislation pertaining to care and use of animals under experimentation.

Clinical, Haematological and Virological Monitoring.

All animals were monitored daily from pre-vaccination through to day 21 post-challenge. Clinical signs were assessed twice based on 10 clinical parameters scored on a scale of 0-3 (Everett et al. (2010) Vet. Microbiol. 142 26-33) Animals were euthanized by lethal injection of anaesthetic when clinical scores reached or approached 15, out of a possible maximum of 30. Leukocyte and platelet counts were monitored every 2-3 days. Leukocyte counts were obtained by flow cytometry analysis of CD45 stained EDTA blood as described previously (Graham et al., 2010). Platelet counts were obtained from platelet rich plasma (PRP) following centrifugation of EDTA blood at 85×g for 1 min. PRP was diluted 1/500 in CellFIX (BD Biosciences, Oxford, UK) and incubated for 10 min at room temperature. An aliquot of PRP was analyzed on a MACSQuant flow cytometer (Miltenyi Biotec, Gergisch Gladbach, Germany) and platelet clouds visualised on logarithmic FSC and SSC scales. Viral RNA was detected in EDTA blood samples collected every 2-3 days; total RNA was extracted from 140 μl of blood using QIAmp Virus RNA Mini Extraction kits as described by the manufacturer (Qiagen, Crawley, UK) and CSFV viral copy numbers were assessed by quantitative RT-PCR (Everett et al., 2010).

Serum Neutralisation Assay.

Blood was collected from pigs every 2-3 days post vaccination/challenge in serum separation tubes (BD) and serum obtained by centrifugation at 1500×g for 10 min. The measurement of CSFV neutralising antibody titres in sera was determined using a standard virus neutralisation peroxidise-linked assay using Alfort 187 CSFV and PK-15 cells (Drew, 2008).

Type I IFN Bioassay.

Type I IFN bioactivity was determined using an Mx/CAT reporter gene assay originally developed for the quantification of bovine IFN-α/β (Fray et al. (2001) J. Immunol. Methods 249 235-244). Serum samples were diluted to 10% (v/v) in serum-free tissue-culture medium and added to reporter bovine kidney cells (MDBK-t2) for 24 hours. A titration of recombinant porcine IFN-α (R&D Systems, Abingdon, UK) was added as a standard. Lysates were prepared from the cultures and CAT enzyme measured by ELISA using an enhanced ABTS substrate (Roche, Welwyn Garden City, UK). Luminescence was read using a FLUOstar OPTIMA microplate reader (BMG Labtech, Aylesbury, UK).

In Vitro Stimulation of Peripheral Blood Cells and Analysis of IFN-γ Production.

Heparinised blood was collected from pigs every 2-3 days post vaccination/challenge. Leukocytes were prepared using a standard protocol. In brief, blood was centrifuged at 800×g for 10 min and visible ‘buffy coat’ material aspirated. Contaminating erythrocytes were lysed by addition of 10 ml of Pharmlyse Buffer (BD Biosciences) and incubation for 10 min at room temperature before being washed three times in Hank's buffered salt solution (HBSS) (Invitrogen, Paisley, UK). Cells were finally resuspended in RPMI-1640 medium supplemented with 10% foetal calf serum (FCS) and antibiotics (all from Invitrogen). Cell densities were determined by analysing 50 μl of cell suspension using a MACSQuant flow cytometer and gating on events with typical SSC and FSC properties. Cell densities were adjusted to 5×10⁶ cells/ml and 100 μl transferred to wells of a 96 well round-bottom or 24 well plate, respectively. Cells were stimulated by the addition of an equal volume of medium containing CSFV or BVDV at a multiplicity of infection (MOI) of 1, 0.1 or 0.01. To serve as a negative control, a mock inoculum prepared from uninfected PK-15 cells was added in an equivalent volume to the viruses.

Alternatively, recombinant proteins and synthetic peptide pools were added at final concentrations of 10 μg/ml and 1 μg/ml, respectively. Plates were then incubated for 72 hours at 37° C. in a humidified 5% CO₂ atmosphere. After incubation, the contents of wells were mixed by repeated pipetting, centrifuged at 400×g for 5 min, cell free supernatants removed and immediately stored at −80° C. until analysed. IFN-γ was measured in the culture supernatants, diluted 1:2 in Standard Diluent Buffer using a swine IFN-γ ELISA according to the manufacturers instructions (Biosource, Invitrogen) and absorbance at 440 nm read using a FLUOstar OPTIMA microplate reader (BMG Labtech, Aylesbury, UK).

Intracytoplasmic IFN-γ and LAMP-1 Degranulation Flow Cytometric Analysis.

Peripheral blood leukocytes were prepared and stimulated with CSFV (MOI=1) or mock supernatant as described above. After 20 hours of culture, Brefeldin A (1 μg/ml; BD Bioscience) was added and cells incubated for a further 4 hours. Cultured cells were harvested and stained with an antibody for porcine CD3 (VMRD, Pullman, USA) and FITC-conjugated anti-CD4 and CD8 mAbs (AbD Serotec) for 10 min at room temperature. CD3 staining was visualised by staining cells for 10 min with an APC-conjugated anti-mouse IgG1 secondary antibody (BD Biosciences).

After treatment with a Cytofix/Cytoperm kit (BD Bioscience), according to the manufacturer's instructions, the cells were stained with fluorochrome conjugated antibodies against perforin (BD Bioscience) and IFN-γ (AbD-Serotec, Oxford, UK) or isotype control antibodies and incubated for 10 min at room temperature. Cells were washed and resuspended in Dulbecco's phosphate buffered saline (dPBS) supplemented with 2% foetal bovine serum (FBS) and analysed on a MACSQuant flow cytometer (Miltenyi Biotec). A lymphocyte gate was defined using forward and side scatter properties. CD4⁺ T cells were identified within the CD4⁺ and CD3⁺ population. CD8⁺ T cells were identified as the CD8^{hi} CD3⁺ population and then two populations were defined on the basis of staining with anti-perforin. Anti-IFN-γ staining was assessed on these gated populations.

To assess LAMP-1 (CD107a) degranulation a modification of the protocol developed by Betts et al., (2003) was used. In brief, porcine PBL were infected with C-strain CSFV or mock supernatant and incubated for 4 hours at 37° C. as described above. Monensin (GolgiStop; BD Bioscienes) and anti-CD107-Alexa Fluor 647 (AbD Serotec) were added to cells at concentrations recommended by the manufacturers and cells incubated for a further 4 hours. During the last 15 min of the incubation cells were stained with anti-CD4-FITC and anti-CD8-PE and then washed twice with FACS buffer before being analysed by flow cytometry.

Assessment of the Effects of IFN-γ on CSFV In Vitro.

Peripheral blood monocytes were isolated from CSFV naïve pigs by staining of PBMC with CD14 microbeads (Miltenyi Biotec) followed by positive magnetic sorting as described by the manufacturer (Miltenyi Biotec). The effects of recombinant porcine IFN-γ (R&D Systems, Abingdon, UK) on CSFV infection and replication was assessed by either pre-treating or post-treating CSFV infected monocytes cultured in ultra-low bind plates (Corning). Recombinant IFN-α (R&D Systems) treated, untreated and uninfected cells were included as positive and negative controls. Cells were pre-treated with recombinant cytokines for 24 hours, after which time the cells were washed and infected with CSFV reference strain Alfort 187 at an MOI of 5. After 2 hours, the virus-containing supernatant was removed, cells were washed and fresh media was added. After a further 24 hours in culture, the amount of viral RNA in the supernatant was assessed by quantitative RT-PCR as described above. Cells were harvested and stained with a fixable violet live-dead stain (Invitrogen) for 10 min at room temperature. Cells were then washed, fixed and permeabilised as described above and stained intracytoplasmically with a mAb specific for CFSV E2 (WH303; Paton et al., 1995) followed by an APC conjugated anti-mouse IgG1 secondary antibody (BD Biosciences). Expression of CSFV E2 was then assessed by flow cytometry.

For post-treatment experiments, monocytes were infected for two hours, washed, treated with recombinant cytokines for 24 hours and analysed as described for the pre-treated cells. To investigate the potential roles of iNOS induction and tryptophan catabolism in IFN-γ mediated anti-CSFV effects, experiments were conducted as described above except that parallel cultures containing 100 μM L-N6-(1-iminoethyl)lysine hydrochloride (Sigma) or 200 μg/ml of D- or L-tryptophan (Sigma) were included.

Statistical Analysis.

ANOVA was used for the analysis of fixed effects on different traits using GraphPad Prism 5 (Prism 5 for Windows, Version 5.01, GraphPad Software, Inc. La Jolla, USA). A plot of the total [log₁₀] viraemia versus the virus-specific IFN-γ response suggested a negative exponential relationship between them, and thus a linear regression was carried out with log total viraemia as the dependent variable and the total virus-specific IFN-γ response as the independent variable.

### Results

Outcome of Vaccination and Challenge with Diverse CSFV Viruses.

The ability of the C-strain vaccine to confer rapid protection was remarkably similar when animals were challenged with the Genotype 2.1 UK2000/7.1 CSFV isolate or the Genotype 3.3 That CBR93 isolate (summarised in Table 1).

TABLE 1
Clinical and virological outcome of challenge with UK2000/7.1
and CBR93 CSFV isolates following C-strain vaccination^{a}

| | | | | Minimum | Minimum | | |
|---|---|---|---|---|---|---|---|
| | | | | leukocyte | platelet | Maximum viral | Proportion |
| CSFV | | Maximum | Maximum | count | count | titre (RNA | of animals |
| challenge | Experimental | temperature | clinical | (×10³ | (×10⁵ | copies/μl | requiring |
| isolate | group | (° C.) | score | cells/μl) | cells/μl) | blood) | euthanasia |
| UK2000 | Day −5 vaccinated | 38.6 | 1.0 | 13.3 | 6.8 | ND^{c} | 0/6 |
| 7.1 | (n = 6) | (37.9-39.5) | (0.3-1.7) | (11.2-15.3) | (4.8-8.7) | | |
| | Day −3 vaccinated | 38.9 | 1.4 | 10.2 | 4.8 | 169 | 0/5 |
| | (n = 5)^{b} | (38.0-39.8) | (0.7-2.1) | (7.8-12.6) | (1.6-8.1) | (166-503) | |
| | Day −1 vaccinated | 39.0 | 5.5 | 5.7 | 4.5 | 9.4 × 10⁵ | 3/6 |
| | (n = 6) | (37.9-40.1) | (2.9-8.1) | (4.1-7.4) | (3.1-5.9) | (0.6-2.5 × 10⁶) | |
| | Unvaccinated | 40.4 | 7.2 | 3.7 | 1.8 | 1.3 × 10⁶ | 4/4 |
| | (n = 4) | (39.4-41.4) | (6.4-8.0) | (1.8-5.5) | (1.2-4.9) | (0.3-2.2 × 10⁶) | |
| CBR93 | Day −5 vaccinated | 38.6 | 0.8 | 10.6 | 4.3 | ND | 0/6 |
| | (n = 6) | (38.2-39.1) | (0-1.6) | (9.5-11.8) | (1.9-6.7) | | |
| | Day −3 vaccinated | 38.2 | 0.6 | 8.9 | 6.0 | 320 | 0/6 |
| | (n = 6) | (37.8-38.6) | (0.5-1.7) | (8.3-9.6) | (4.6-7.5) | (321-962) | |
| | Day −1 vaccinated | 39.3 | 5 | 7.6 | 5.1 | 6.8 × 10⁵ | 2/6 |
| | (n = 6) | (38.5-40.1) | (2.0-8.0) | (5.4-9.8) | (2.4-7.9) | (0.8.5-2.2 × 10⁶) | |
| | Unvaccinated | 40.2 | 7 | 6.4 | 2.5 | 3.0 × 10⁶ | 4/4 |
| | (n = 4) | (39.6-40.9) | (5.2-8.8) | (5.8-7.0) | (7.8-5.8) | (0.4-5.5 × 10⁶) | |

^{a}Mean values are presented with 95% confidence interval shown in parentheses.
^{b}One animal in this experimental group developed a bacterial pneumonia only two days post-CSFV challenge and was therefore excluded from the analyses.
^{c}ND: not detected

Vaccination of pigs five days prior to challenge with either virus prevented the onset of clinical signs, leukocyte and platelet counts remained normal and no viral RNA was detected in blood. In the groups that received the vaccine three days prior to challenge, one animal displayed signs of a bacterial pneumonia only two days post-challenge and was excluded from the analyses. Of the remaining animals, none developed significant clinical symptoms, they showed a mild and transient drop in leukocyte and platelet counts; a low level viraemia was detected in some animals around day 6-15 post-challenge. In the groups that were challenged one day after vaccination, there was a clear dichotomy in the outcome to challenge. Three pigs that were challenged with UK2000/7.1 CSFV and two pigs challenged with CBR93 CSFV developed clinical CSF, displayed both leukopenia and thrombocytopenia and exhibited a viraemia that continued to rise (>1×10⁶ RNA copies/μl blood) until a point at which the animals were euthanized for welfare reasons. The remaining animals were able to control the infection and had mild clinical signs that receded, transient leukopenia and thrombocytopenia and viraemia that peaked on day 9 post-challenge before declining. All the challenge control pigs developed clinical CSFV requiring euthanasia by day 17 post-challenge.

In the animals vaccinated with one of the epitope polypeptides SEQ ID NOs:1, 2 or 3, there was reduced occurrence of symptoms of CSFV infection as compared with the animals in the control group.

Serum Antibody and Type I IFN Responses do not Associate with Vaccine Induced Rapid Protection.

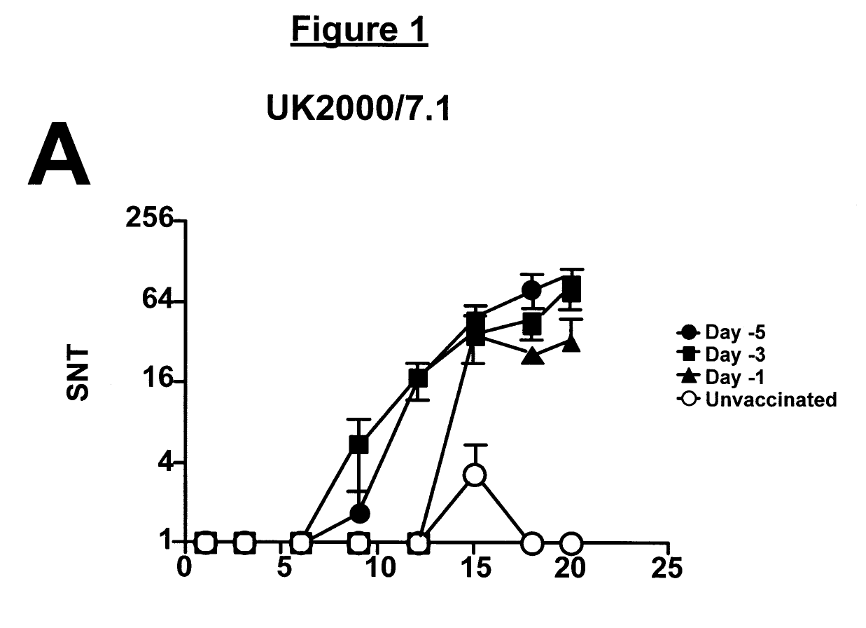

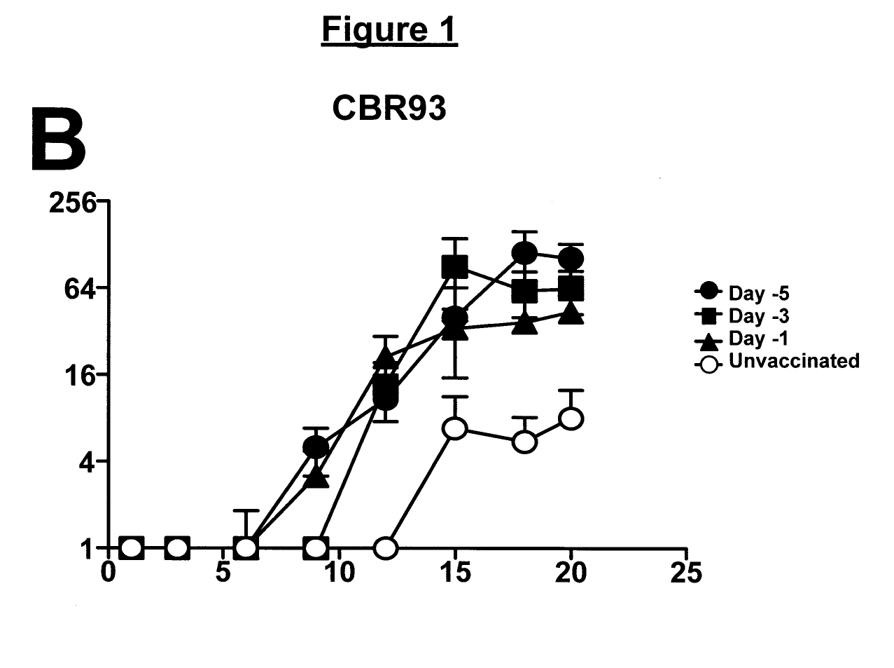

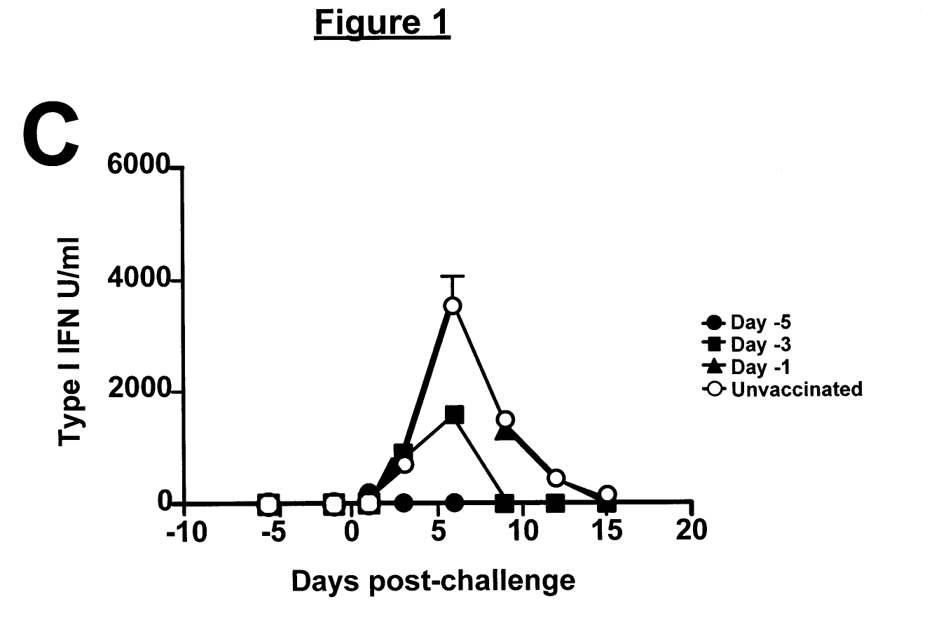

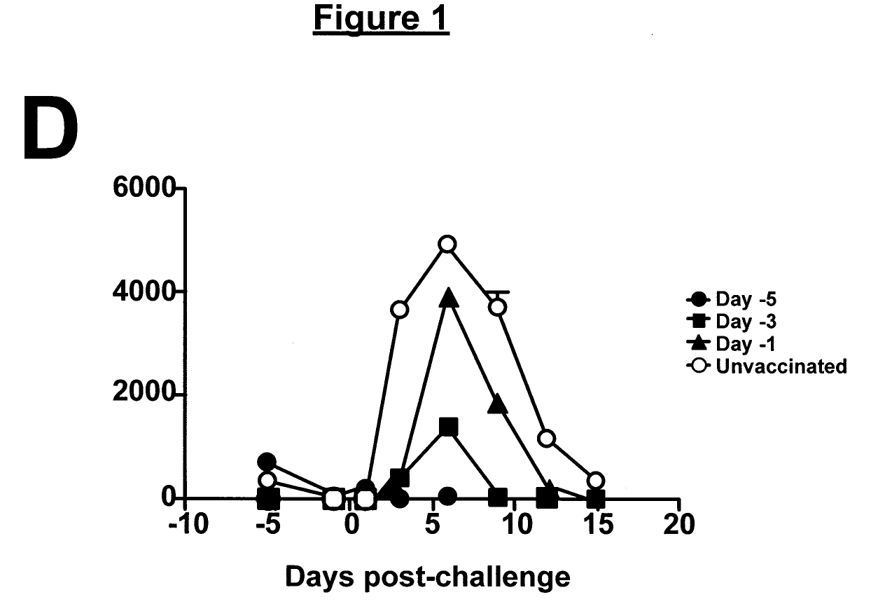

Since both neutralising antibody and type I IFN responses may exert potent inhibitory effects on CSFV, these responses were investigated in the sera of vaccinated and challenged pigs (FIG. 1 showing mean data, error bars represent SEM). Regardless of when animals were vaccinated, significant virus neutralising antibody titres were only consistently detected after day 12 post-challenge (p<0.05). The animals vaccinated one day prior to UK2000/7.1 challenge did not show significant neutralising titres until day 15. Unvaccinated animals challenged with the UK2000/7.1 virus did not mount a significant antibody response while those challenged with CBR93 responded after 15 days (FIGS. 1A and B; p<0.05). Pigs vaccinated on Day-5 had a small but significant type I IFN response detectable only on Day 1 post-challenge (p<0.05). Animals vaccinated on Day-3 had type I IFN detectable in serum between days 3-6 post-challenge whereas day-1 vaccinates and unvaccinated animals had the highest responses, detectable from day 3 until day 9 post-challenge (FIGS. 1C and D; p<0.05).

Virus-Specific T Cell IFN-γ Responses Correlate with Vaccine Induced Rapid Protection.

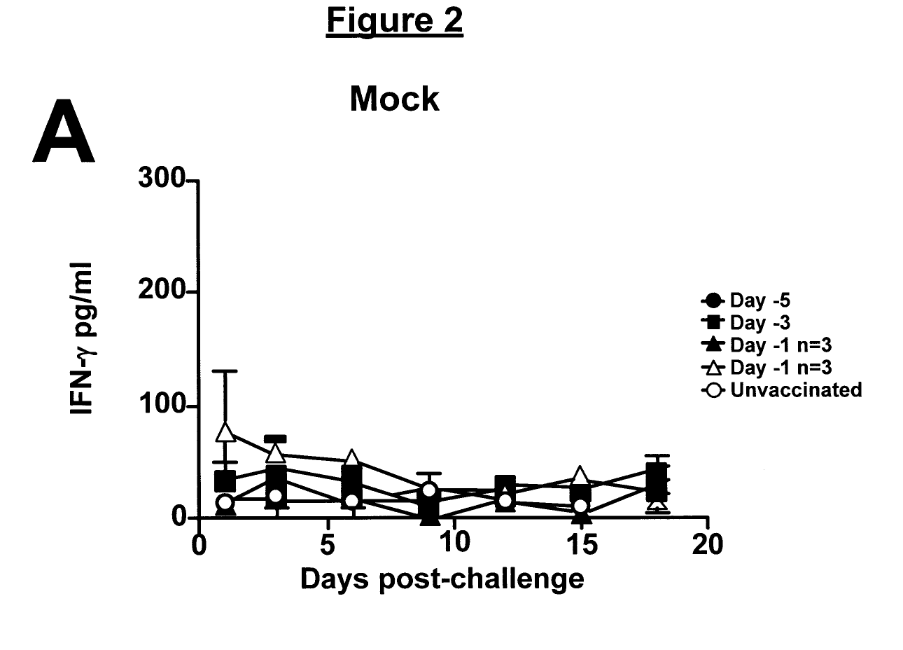

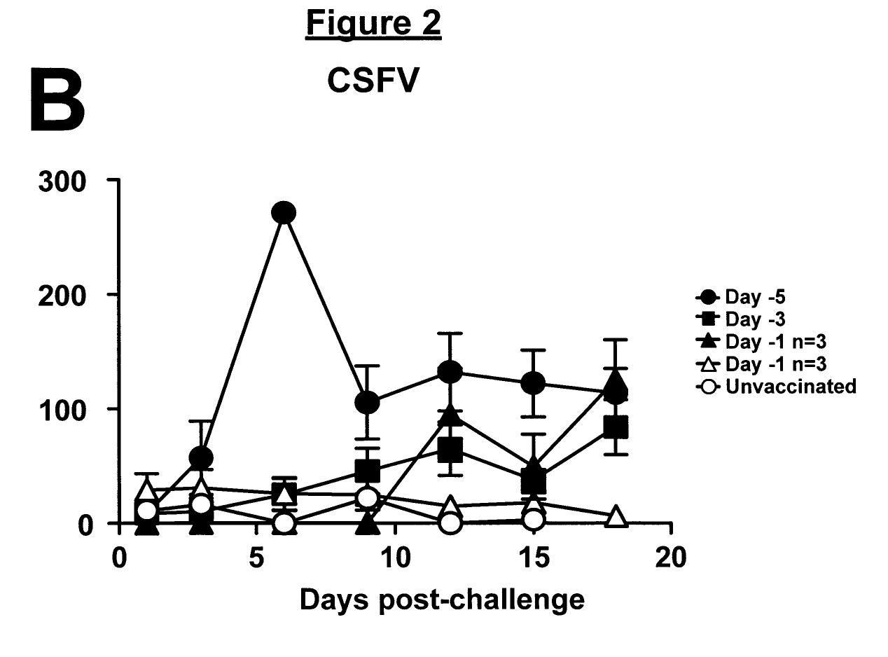

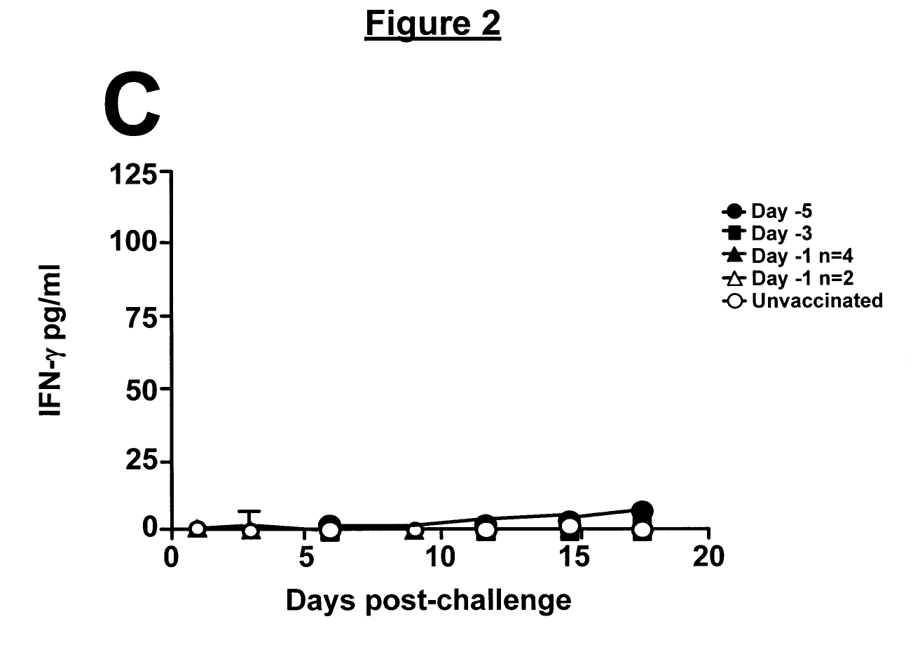

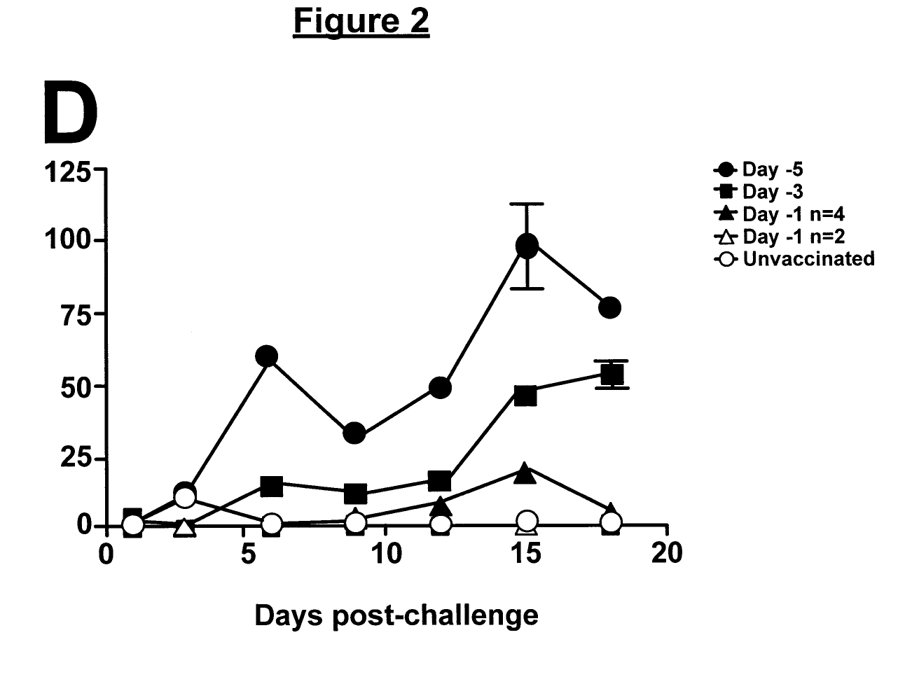

Virus-specific T cell IFN-γ responses were measured longitudinally following in vitro stimulation of peripheral blood leukocytes with the live-C-strain virus or a mock inoculum (FIG. 2, showing mean data, error bars represent SEM). In the UK2000/7.1 trial, IFN-γ responses of Day-5 vaccinates, following in vitro stimulation with C-strain virus, peaked 6 days post-challenge (11 days post-vaccination) and remained elevated (FIGS. 2A & B). Virus specific IFN-γ responses of Day-3 vaccinates and the Day-1 vaccinates that controlled the challenge infection, were detected 9 days post-challenge (12 days post-vaccination) and 12 days post-challenge (13 days post-vaccination), respectively. These responses were significantly reduced compared to the Day-5 vaccinates (p<0.05). No virus specific IFN-γ responses were detected from either the Day-1 vaccinated animals that succumbed to the challenge infection, or the unvaccinated challenge control pigs.

In the CBR93 trial, the Day-5 vaccinates again displayed the strongest virus-specific IFN-γ response detectable from Day 6 post-challenge (p<0.01) (FIGS. 2C & D). The Day-3 vaccinates also mounted an IFN-γ response that was detected, albeit at a lower magnitude from Day 6 post-challenge. The four Day-1 vaccinates that recovered from the challenge infection had a detectable IFN-γ response from Day 12 post-challenge while the remaining Day-1 vaccinates and the unvaccinated control animals again did not make a detectable virus specific IFN-γ response.

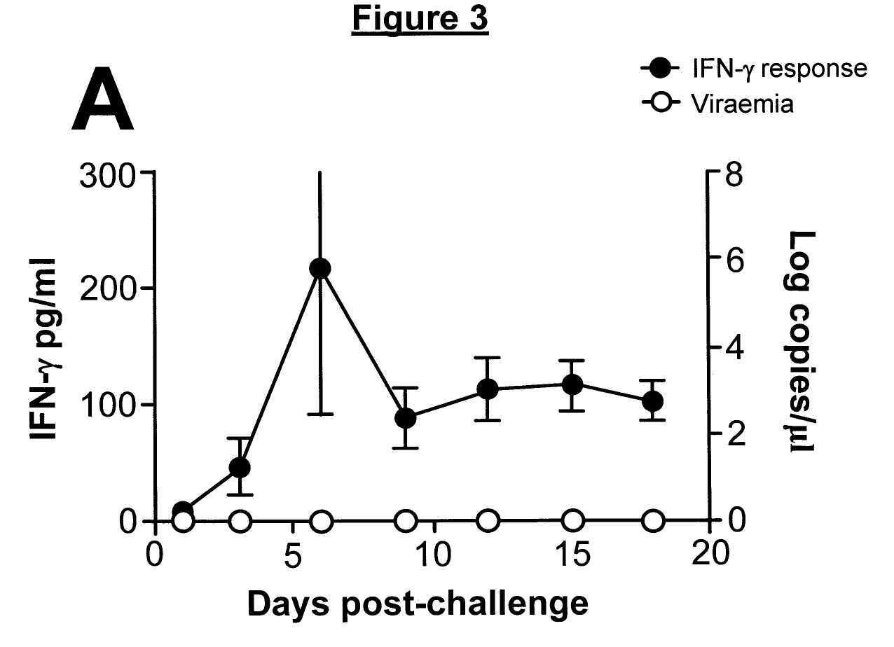

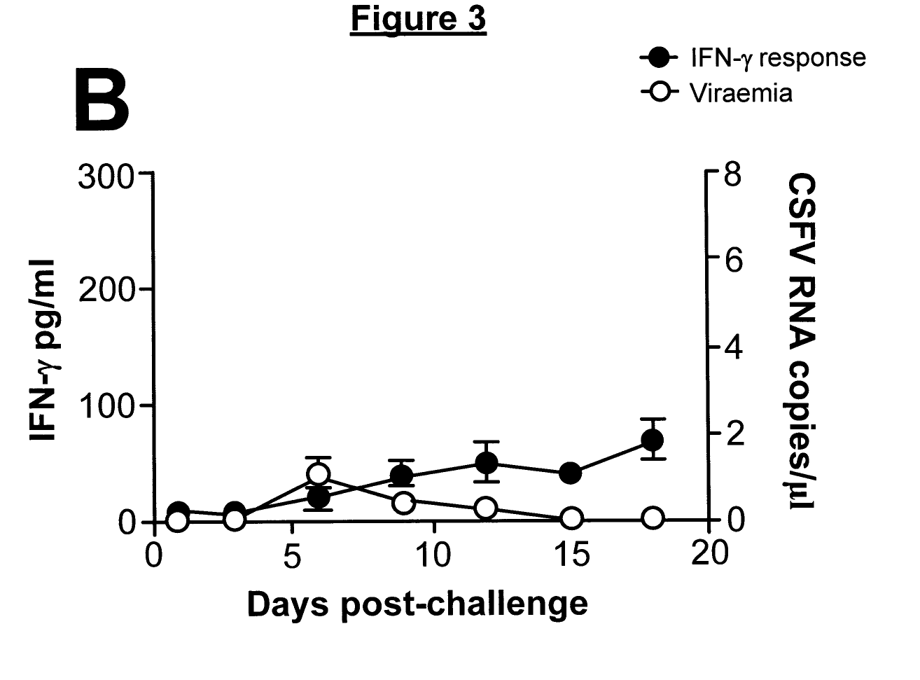

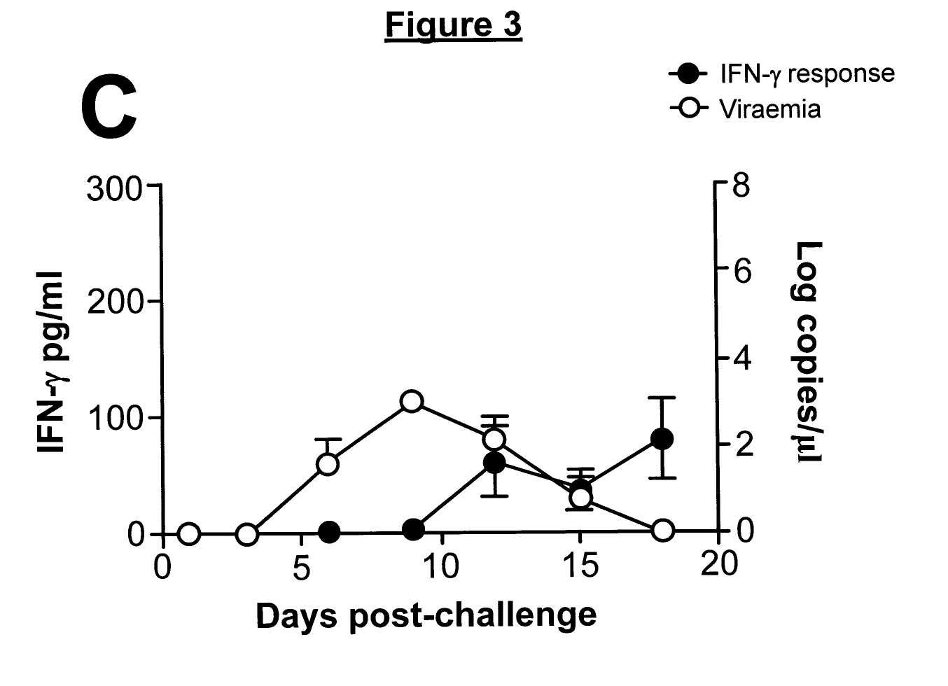

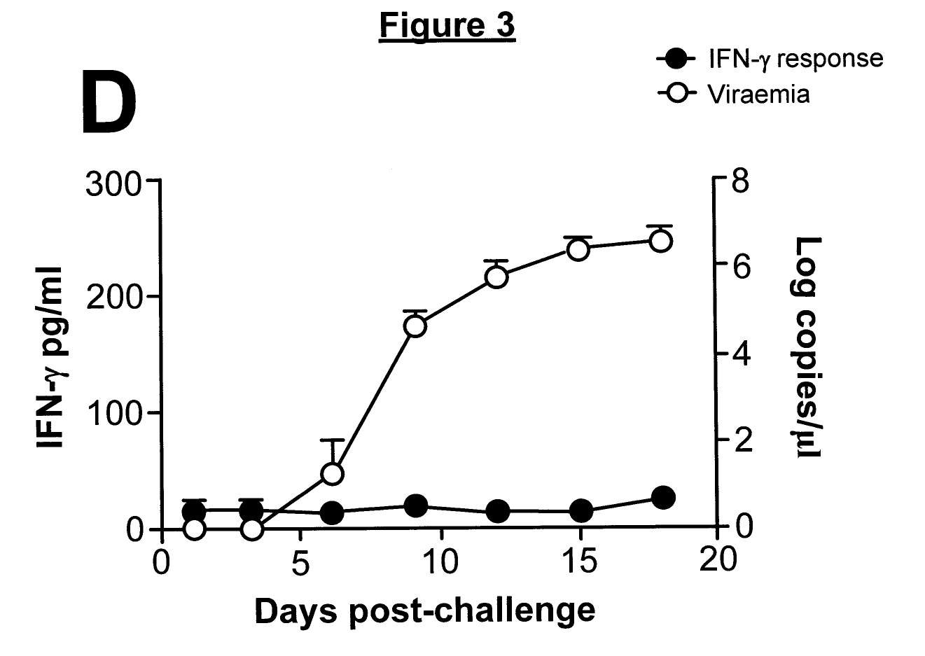

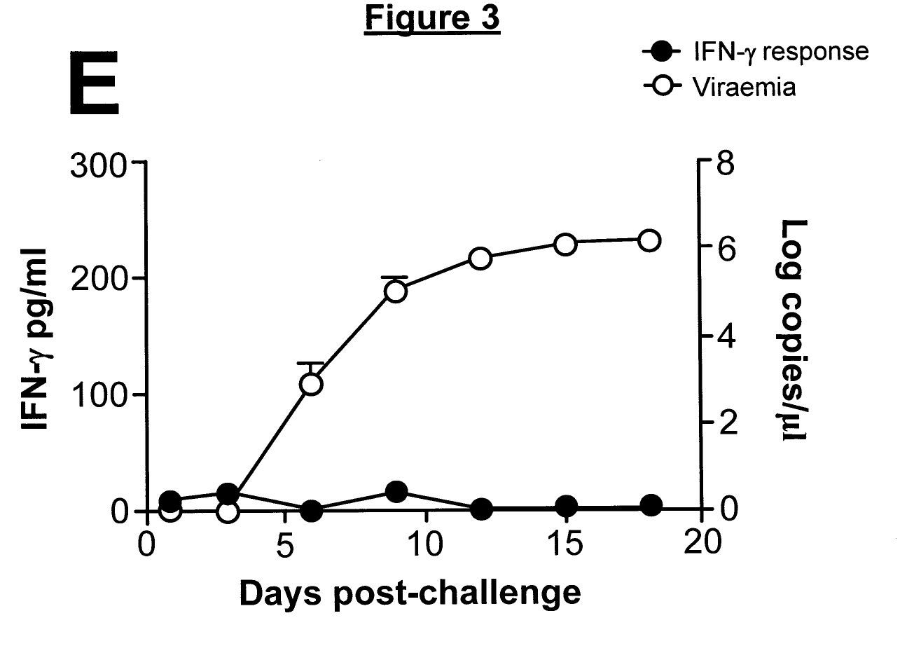

The significance of the IFN-γ T cell responses became further apparent when they were correlated with the viraemia detected in these animals (FIG. 3, showing mean data, error bars represent SEM). A log-linear regression showed a negative exponential relationship between the total [log 10] viraemia and the virus-specific IFN-γ response (p<0.05). The Day-5 vaccinates had robust IFN-γ responses and undetectable viraemia (FIG. 3A), whereas the Day-1 (FIG. 3D) and unvaccinated (FIG. 3E) animals that succumbed to challenge had undetectable IFN-γ responses and uncontrolled viraemia. Interestingly, the decline and disappearance of viraemia in the Day-3 and Day-1 vaccinates, that controlled the challenge infection, occurred coincidentally with the appearance of virus specific IFN-γ responses.

### T Cell IFN-γ Responses are Directed Against Highly Conserved Epitopes on Both Structural and Non-Structural Proteins.

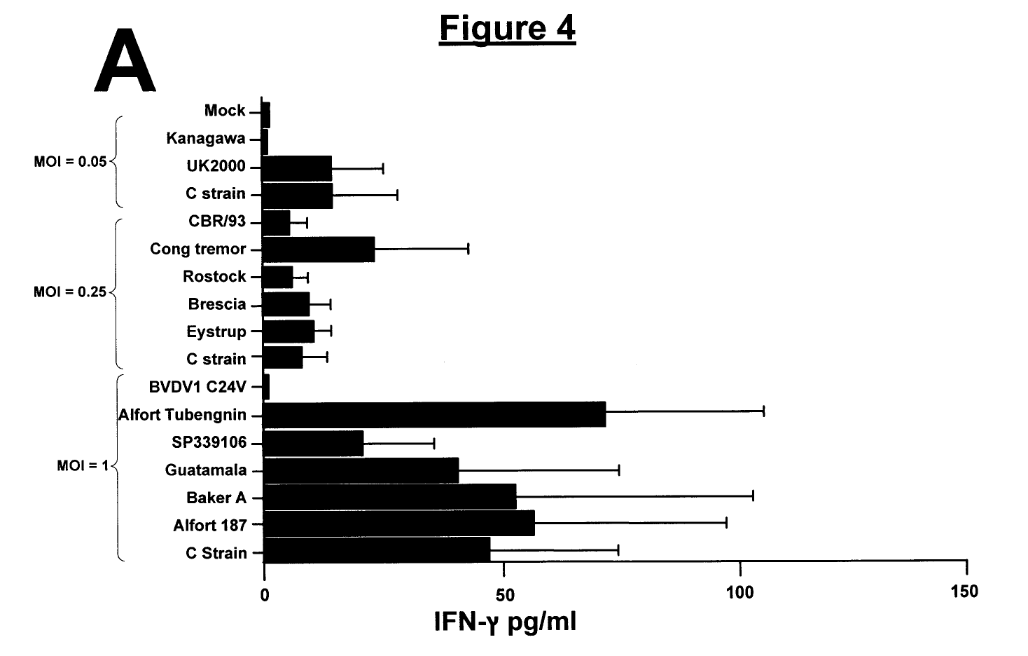

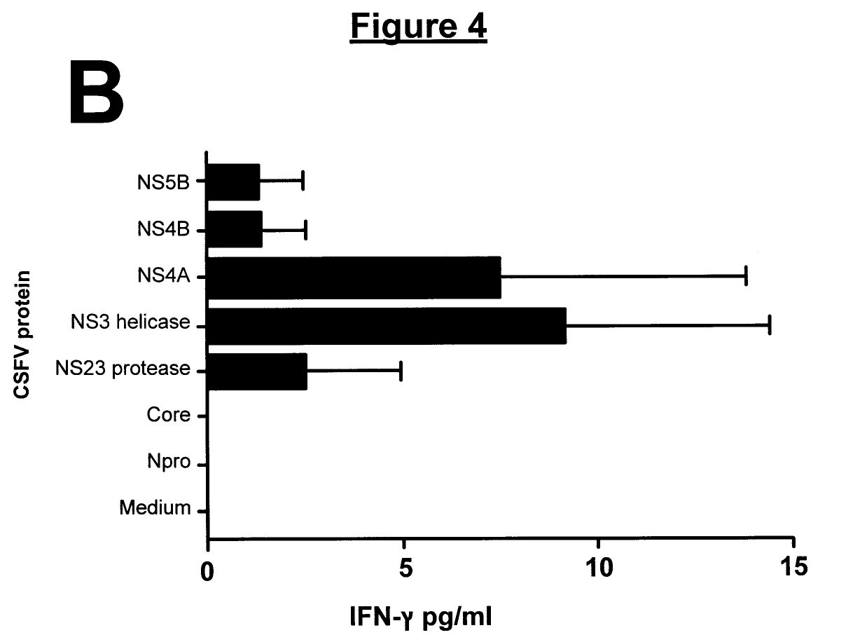

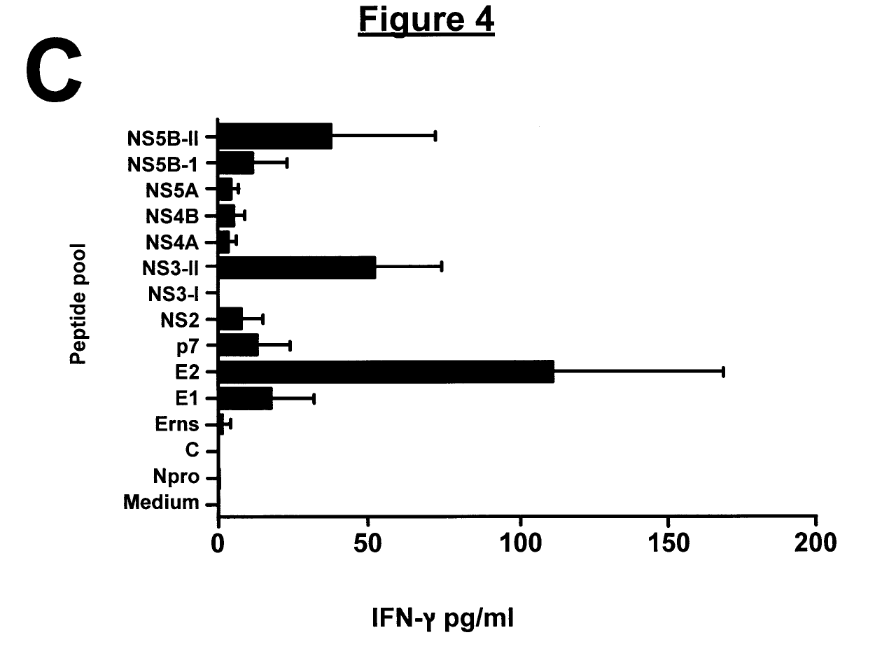

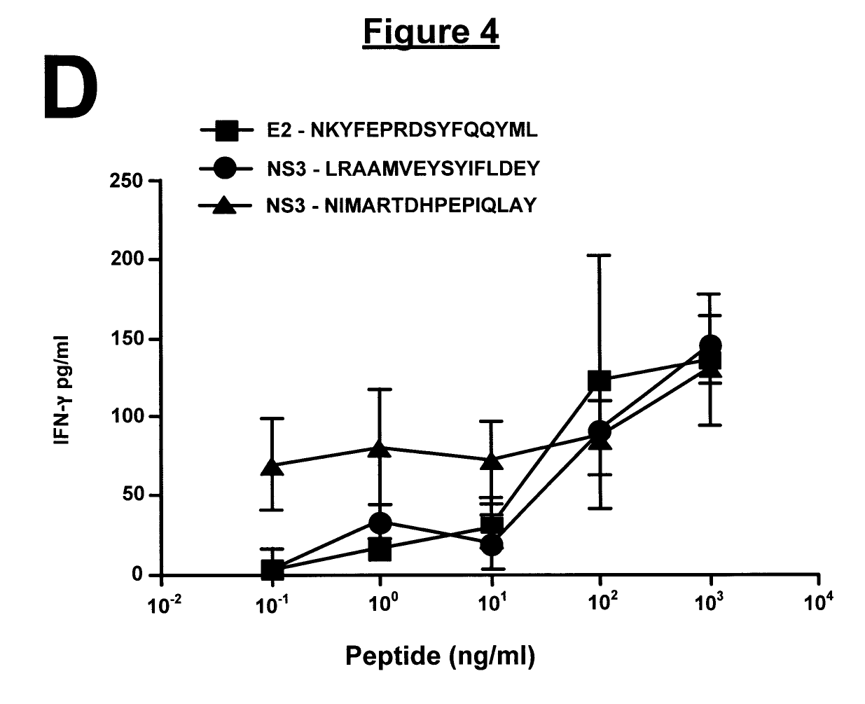

The specificity of the IFN-γ responses of pigs following vaccination and challenge was further addressed by first assessing responses following PBMC stimulation with a panel of 14 viruses, including representatives of 9 of the 10 sub-genotypic clusters of CSFV as well as BVDV (FIG. 4, showing mean data, error bars represent SEM). Due to limitations with obtaining optimal titres of all virus stocks, the viruses were tested at the optimal MOI of 1, or at an MOI of 0.25 or 0.05 (FIG. 4A). Animals mounted IFN-γ responses to all viruses tested except against the Japanese Genotype 3.4 Kanagawa isolate and the Oregon C24V strain of BVDV.

The specificity of these cross-strain specific responses was then probed by assessing responses against a panel of seven recombinant CSFV proteins (FIG. 4B). No responses were measured against the Npro or core proteins but were after exposure to the non-structural proteins NS2/3 protease, NS3 helicase domain, NS4a, NS4B and NS5B, although the dominant responses appeared to be directed against the C-terminus helicase domain of NS3 and the NS4A protein.

Cross-reactive epitopes were mapped by taking advantage of an overlapping synthetic peptide library that represented the polyprotein of BVDV. Screening of peptide pools representing viral proteins or portions of larger proteins against PBMC from vaccinated and challenged pigs, revealed significant IFN-γ responses against a number of peptide pools (FIG. 4C). The dominant responses were observed against peptide pools representing the structural E2 protein, the helicase domain of NS3 (NS3-II) and to the C-terminal portion of NS5B (NS5B-II). Antigenic peptides were then identified from the E2 and NS3-II peptide pools by screening titrations of individual peptides against PBMC from an individual animal. This analysis showed recognition of a single antigenic peptide from E2, NKYFEPRDSYFQQYML (SEQ ID NO:1), and to two distinct peptides from NS3, LRAAMVEYSYIFLDEY (SEQ ID NO:2) and NIMARTDHPEPIQLAY (SEQ ID NO:3) (FIG. 4D). Bioinformatic analyses showed that the antigenic peptide sequences were conserved in the C-strain, UK2000/7.1 and CBR93 CSF viruses, as well as other CSFV isolate sequences deposited in GenBank (data not shown).

### Investigation of the Role of IFN-γ as a Marker of Cytotoxic T Cell Responses or an Effector Molecule Against CSFV.

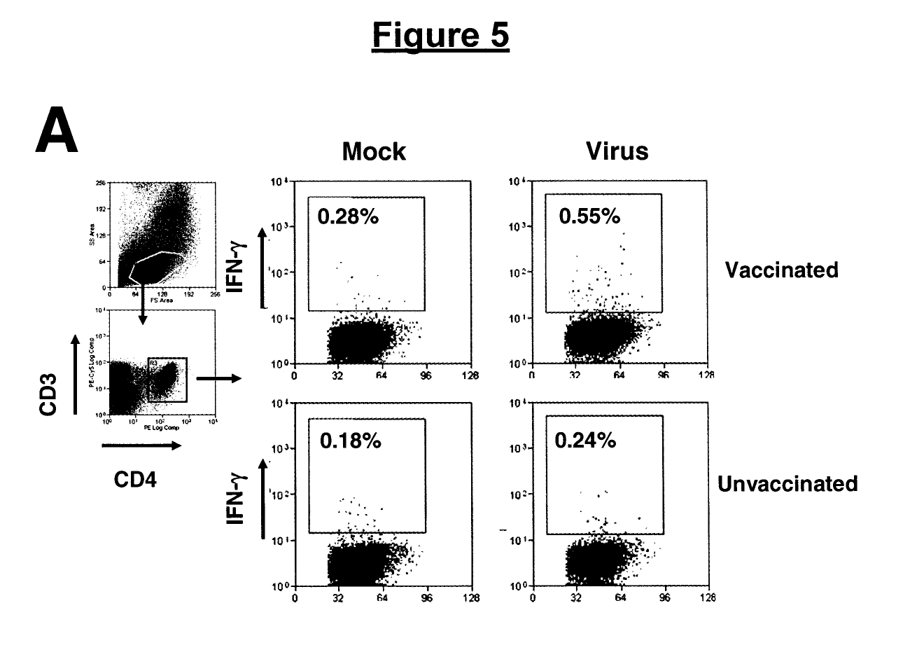

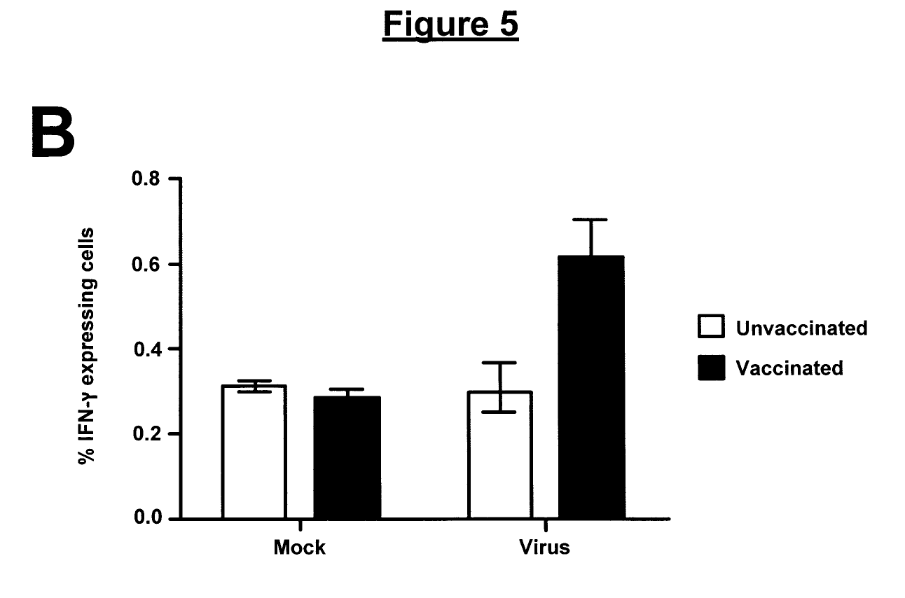

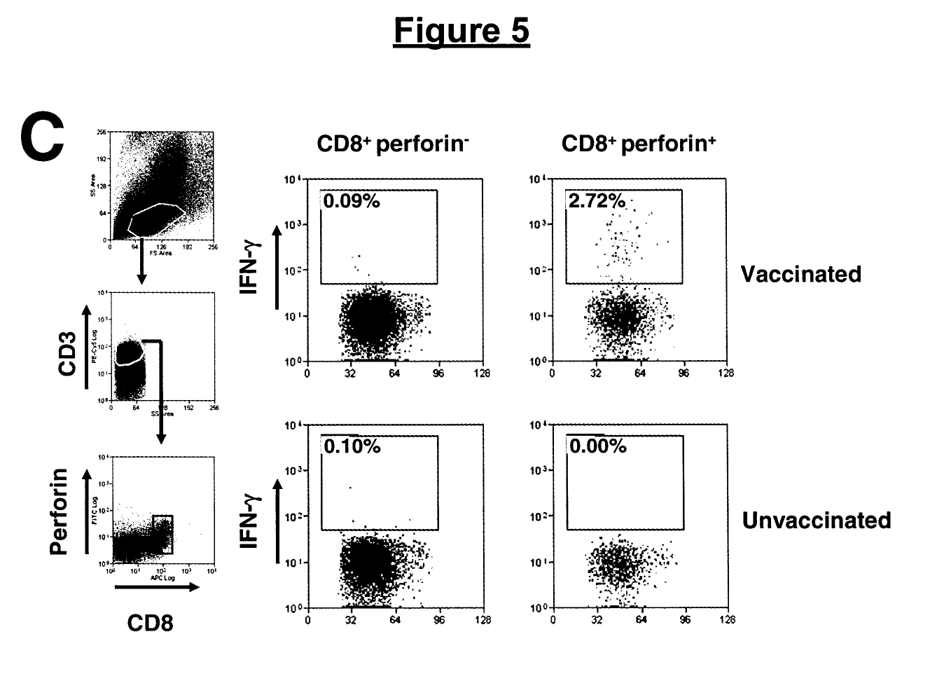

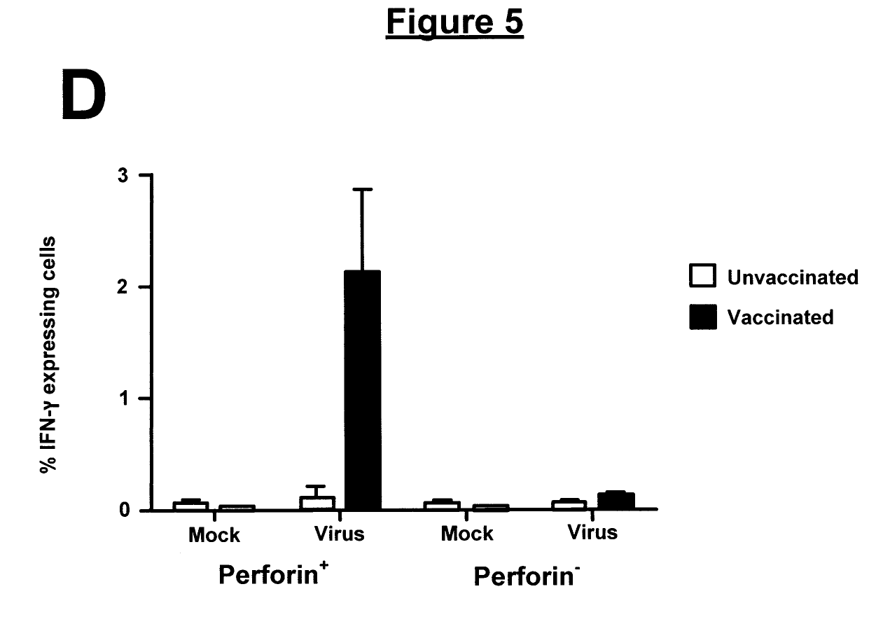

To better understand the significance of IFN-γ responses in the protection against CSFV, flow cytometric studies were undertaken to phenotype the virus-specific IFN-γ secreting cells (FIG. 5, showing mean data, error bars represent SEM). Staining of CD4⁺CD3⁺ T cells for IFN-γ production following mock or CSFV stimulation showed a doubling in the frequency of IFN-γ⁺CD4 T cells (−0.6% of CD4 T cells) in CSFV stimulated cultures from vaccinated animals compared to mock-stimulated or virus-stimulated cultures from unvaccinated animals (FIGS. 5A & B). Staining of CD8^{hi}CD3⁺ T cells with anti-perforin antibodies in conjunction with staining for IFN-γ, revealed that ˜2% of perforin CD8 T cells from vaccinated animals produced IFN-γ in response to virus stimulation. The frequencies of IFN-γ secreting perforin⁻ CD8 T cells was <0.1% and comparable to those observed following mock stimulation or virus stimulation of CD8 T cells from unvaccinated animals (FIGS. 5C & D; p<0.05)).

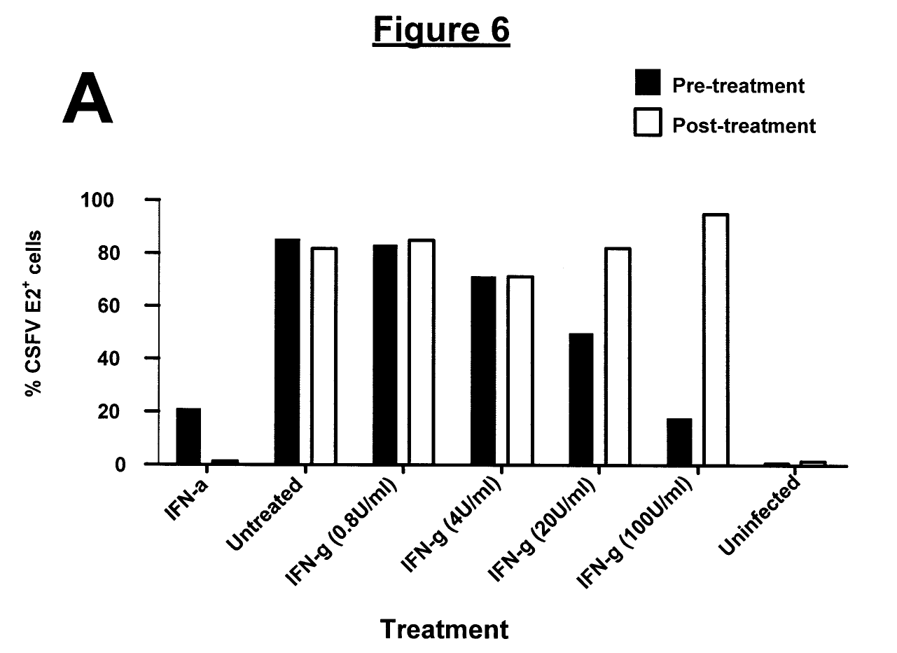

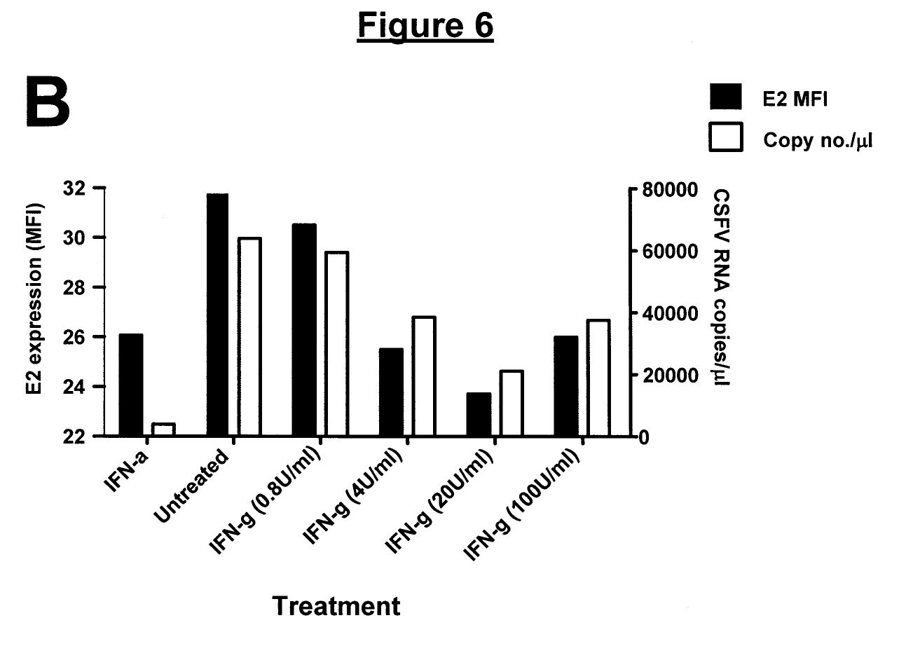

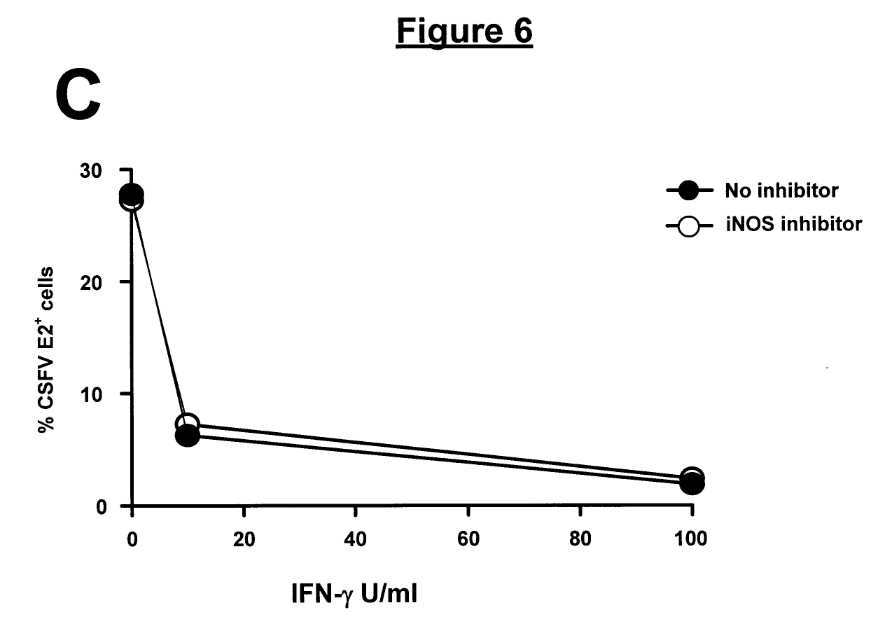

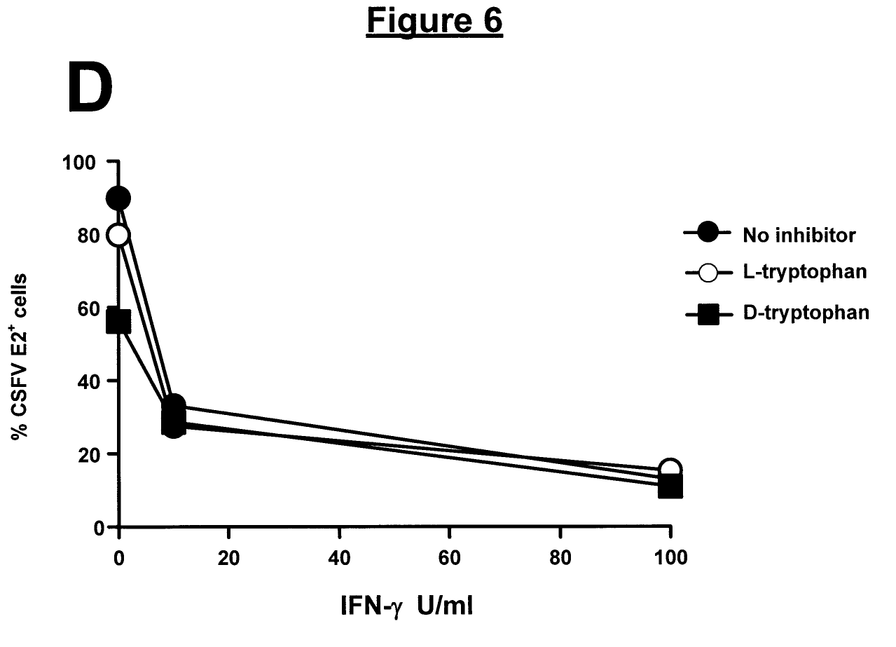

Finally, the inventor tested whether IFN-γ itself may be directly involved by exerting inhibitory effects on CSFV. This was addressed using an in vitro infection model, where porcine monocytes were treated with recombinant IFN-γ either before or after infection with CSFV and the effects on the proportion of infected cells, the levels of E2 protein present in infected cells and the amount of viral RNA in culture supernatants assessed. Pre-treatment of monocytes with a titration of recombinant IFN-γ prior to infection resulted in a dose-dependant reduction in the proportion of CSFV infected cells after 24 hours (FIG. 6A). Treatment of cells with 100 U/ml IFN-γ reduced the number of infected cells down to a level comparable with those of cells pre-treated with IFN-α. IFN-γ treatment following CSFV infection had no effect on the proportion of infected cells whereas IFN-α treatment eliminated the infection (FIG. 6A). However, post-infection treatment with IFN-γ did have a dose-dependant effect on the reduction in the levels of viral E2 protein in the cytosol of infected cells and viral RNA released into the culture supernatant (FIG. 6B).

The potential roles of nitric oxide production or the depletion of tryptophan in the inhibitory effects mediated by IFN-γ, were investigated under the post-infection treatment condition. Infected cells were treated with IFN-γ in the presence or absence of the specific iNOS inhibitor L-N-6-(1-iminoethyl)lysine hydrochloride, or L- or D-tryptophan (FIGS. 6C & D). After 48 hours, there was an IFN-γ mediated reduction in the proportion of CSFV infected cells, which was unaffected by the presence of the iNOS inhibitor or tryptophan. The inhibitors similarly had no effect on the levels of E2 expression by infected cells or the amounts of CSFV RNA detectable in culture supernatants (data not shown).

### Discussion

It has previously been reported that CSFV C-strain can protect pigs from the clinical signs of disease within seven days of vaccination and that this may precede the appearance of virus-neutralising serum antibodies but not T cell responses (van Oirschot 2003). However, the present work provides, for the first time, a detailed vaccination study that convincingly shows a correlation between the induction of T cell IFN-γ responses and the protection afforded. It also highlights that virulent CSFV subverts the induction of the T cell IFN-γ response, which would otherwise be a potent anti-CSFV mechanism. In this study the inventor has shown that C-strain vaccination five days prior to challenge provided complete protection and vaccination three days prior to challenge protected against disease although low level viraemia was present in some of the animals.

Vaccination, regardless of the period prior to challenge, induced neutralising antibody responses after 12-15 days post-challenge, which agrees with previous data obtained following C-strain vaccination. There was no association between antibody titres and the protection conferred by vaccination. CSFV is able to inhibit the induction of type 11FN responses through the degradation of IRF3 by viral Npro (Bauhofer et al. (2007) J. Virol. 81 3087-3096) although the virus remains sensitive to the antiviral effect of Type I IFN in vitro (Xia et al. (2005) Vet. Immunol. Immunopathol. 104 81-89). By virtue of their signalling through IRF7 as opposed to IRF3, plasmacytoid dendritic cells (pDC) retain their capacity to produce type I IFN in response to CSFV and pDC have been attributed as the source of type I IFN in sera early after CSFV infection (Tarrada et al. (2010) Vet. Microbiol. 142 51-58), which correlates with depletion of lymphocytes, leading to speculation that this response may be detrimental rather than beneficial to the pig. Since it has been shown that C-strain Reims CSFV is similarly not impaired in its ability to block Type I IFN responses from monocytic cells (Ruggli et al. (2003) J. Virol. 77 7645-7654), it is perhaps not surprising that a Type I IFN response associated with vaccination was not detected, but rather serum Type I IFN responses post-challenge were observed that were inversely correlated with protection.

Other reports of rapid vaccine-induced T cell mediated protection are scant in the literature. Protection of cattle against bovine herpes virus 1 (BHV-1) has been shown within 5 days post-vaccination and was similarly associated with virus specific IFN-γ production (Woolums et al. (2003) Vaccine 21 1158-64). Given that rapid vaccine protection would be a major asset in the control of epizootic/epidemic diseases or even biological weapons attacks, it is surprising that others have not evaluated protection in the period preceding the appearance of antibody. The broad cross-reactivity of the T cell responses correlates well with the observation that C-strain CSFV may provide protection against virtually any heterologous CSFV (Aynaud, (1988) Classical swine fever and related viral infections. ISBN 0898389690; Martinus Nijhoff Publishers, Boston, USA. pp 165-180).

While cytotoxic T cell responses have been demonstrated following C-strain vaccination (Rau et al., 2006), and may play a role in immune protection, CTL activity was not detected directly in peripheral blood. However, this may be due to low precursor frequency and assay sensitivity since the frequencies of virus-specific T cells, as assessed by IFN-γ staining and flow cytometry, were low. However, the inventor was able to show, in an in vitro model, that CSFV is sensitive to the effects of IFN-γ. Similar to the current findings, it has been shown that IFN-γ inhibits protein synthesis and RNA replication of hepatitis C virus (HCV) replicons and that this inhibitory action is not dependent upon production of nitric oxide nor the catabolism of tryptophan (Frese et al. (2002) Hepatology 35 694-703). More recently it has been shown that the effect of IFN-γ on HCV is dependent upon the RAS-MAPK signalling pathway which may modulate the phosphorylation of non-structural protein (NS)5A (Huang et al. (2005) Hepatology 43 81-90). It is possible that a similar mechanism is responsible for the sensitivity of CSFV to IFN-γ. Interestingly, HCV NS5A has been shown, in a transgenic mouse model, to impair IFN-γ production (Kanda et al. (2009) J. Virol. 83 8463-8469).

The inventor has previously shown that C-strain CSFV induces virus specific IFN-γ responses, whilst these responses are absent in animals experimentally infected with virulent CSFV (Graham et al., 2010). The present data suggest that virulent CSFV is capable of inhibiting the IFN-γ T cell responses induced by C-strain vaccination. Given that, in vitro, both virulent and attenuated CSFV can be used to infect cells resulting in recall T cell IFN-γ responses, it is unlikely that CSFV is directly interfering with the ability of an antigen presenting cell to present viral epitopes nor the ability of primed T cells to mount an IFN-γ response. Rather, it appears that the virus is interfering with the induction of these responses in vivo. Previous studies of the interaction of CSFV with dendritic cells in vitro have used monocyte-derived dendritic cells (MoDC) as a model system and found that CSFV did not effect the phenotypic or functional characteristics of these cells, the exception being the interference with the type I IFN response (Carrasco et al. (2004) J. Gen. Virol. 85 1633-1641). Interestingly, in vivo data early after CSFV infection showed maturation and activation of dendritic cells in the tonsil, blood and spleen (Jamin et al. (2007) Vet. Res. 39 (1):7 Epub 2007 Nov. 20). An analogous scenario occurs with HCV, where the virus does not appear to affect the function of MoDC but induced the upregulation of maturation markers on purified human myeloid DCs (mDCs). However, HCV was able to inhibit the ability of mature mDCs to activate naïve CD4 T cells (Liang et al. (2009) J. Virol. 83 5693-5707).

In conclusion, these studies demonstrate that vaccination of pigs with attenuated CSFV results in broadly cross-reactive IFN-γ secreting T cell responses which are capable of providing rapid protection against virulent CSFV. This potent anti-viral response appears to be inhibited by virulent CSFV thus facilitating its survival and propagation.

1. An isolated polypeptide comprising an amino acid sequence of any one of SEQ ID NOs:1, 2 and 3 or a functional fragment thereof, or comprising an amino acid sequence at least 65% identical to any one of SEQ ID NOs:1, 2 or 3.

2. The polypeptide of claim 1, wherein said polypeptide consists of the amino acid sequence SEQ ID NO:1 or a functional fragment thereof.

3. The polypeptide of claim 1, wherein said polypeptide consists of the amino acid sequence SEQ ID NO:2 or a functional fragment thereof.

4. The polypeptide of claim 1, wherein said polypeptide consists of the amino acid sequence SEQ ID NO:3 or a functional fragment thereof.

5. An isolated polynucleotide encoding at least one polypeptide of claim 1.

6. A vaccine preparation comprising a polypeptide of claim 1.

7. A vaccine preparation comprising a polynucleotide of claim 5.

8. A method of rapidly vaccinating an animal against infection by a pestivirus comprising administering to the animal an effective amount of a polypeptide comprising an E2 epitope polypeptide and/or an NS3 epitope polypeptide.

9. The method of claim 8, wherein the E2 epitope polypeptide comprises SEQ ID NO:1 and/or a functional fragment thereof and/or an amino acid sequence at least 65% identical to SEQ ID NO:1 and wherein the NS3 epitope polypeptide comprises SEQ ID NO:2 and/or 3 and/or a functional fragment thereof and/or an amino acid sequence at least 65% identical to any one of SEQ ID NOs:2 or 3.

10. The method of claim 8, wherein the animal is protected from infection from at least 1 day after administration of the polypeptide.

11. The method of claim 8, wherein the animal is protected from infection from at least 3 days after administration of the polypeptide.

12. The method of claim 8, wherein the animal is protected from infection from at least 5 days after administration of the polypeptide.

13. The method of claim 8, wherein the pestivirus is selected from Classical Swine Fever Virus (CSFV) or Bovine Diarrhoea Virus (BVDV) or Border Disease Virus (BDV).

14. The method of claim 8, wherein the animal is a cetartiodactyla mammal.

15. The method of claim 14, wherein the mammal is a pig, cow, or sheep.

16. A method of rapidly vaccinating an animal against infection by a pestivirus comprising administering to the animal an effective amount of a vaccine preparation of claim 6.

17. A method of rapidly vaccinating an animal against infection by a pestivirus comprising administering to the animal an effective amount of a vaccine preparation of claim 7.

18. A method of determining whether an animal has been exposed to infection by a pestivirus comprising contacting a sample of cells with a polypeptide comprising an E2 epitope polypeptide and/or an NS3 epitope polypeptide and determining whether levels of IFN-γ are increased in said cells after the contacting, an increase in IFN-γ levels being indicative of exposure of the animal to the virus.

19. The method of claim 18, wherein the E2 epitope polypeptide comprises SEQ ID NO:1 and/or a functional fragment thereof and/or an amino acid sequence at least 65% identical to SEQ ID NO:1 and wherein the NS3 epitope polypeptide comprises SEQ ID NO:2 and/or 3 and/or a functional fragment thereof and/or an amino acid sequence at least 65% identical to any one of SEQ ID NOs:2 or 3.

20. The method of claim 18, wherein the pestivirus is selected from Classical Swine Fever Virus (CSFV) or Bovine Diarrhoea Virus (BVDV) or Border Disease Virus (BDV).

21. The method of claim 18, wherein the animal is a cetartiodactyla mammal.

22. The method of claim 21, wherein the mammal is a pig, cow or sheep.


SEQUENCE LISTING (4 sequences)

SEQ ID NO: 1 - protein, 16 aa, Bovine Viral Diarrhoea Virus
NKYFEPRDSYFQQYML

SEQ ID NO: 2 - protein, 16 aa, Bovine Viral Diarrhoea Virus
LRAAMVEYSYIFLDEY

SEQ ID NO: 3 - protein, 16 aa, Bovine Viral Diarrhoea Virus
NIMARTDHPEPIQLAY

SEQ ID NO: 4 - protein, 3898 aa, Bovine Viral Diarrhoea Virus
MELITNELLYKTYKQKPVGVEEPVYDQAGNPLFGERGAIHPQSTLKLPHKRGERNVPTDLASLPKRGDCRSGNSKGPVSGIYLKPGPLFFQDYKGPVYHRAPLELFEEGSMCETTKRIGRVTGSDGKLYHIYVCIDGCITVKSATRDHQRVLRWVHNRLNCPLWVTSCSDTKEEGATKKKQQKPDRLEKGRMKIVPKESERDSKTKPPDATIVVDGVKYQVKKKGKVKSKNTQDGLYHNKNKPPESRKKLEKALLAWAILAMVLIQATMGENITQWNLQDNGTEGIQRAMFKRGVNRSLHGIWPEKICTGVPSHLATDVELKTIHGMMDASEKTNYTCCRLQRHEWNKHGWCNWYNIEPWILIMNRTQANLTEGQPPRECAVTCRYDRDSDLNVVTQARDSPTQLTGCKKGKNFSFAGILMRGPCNFEIAASDVLYKEHDCTGMFQDTAHYLVDGMTNSLESARQGTAKLTTWLGKQLGRLGKKLENKSKTWFGAYAASPYCDVDRKLGYIWFTKNCTPACLPKNTKIIGPGKFDTNAEDGKILREMGGHLSEVLLLSLVVLSDFAPETASAMYLTLHFSIPQSHVDITDCDKTQLNLTVELTTADVIPGSVWNLGKYVCIRPDWWPYETAAAMAFEEVGQVIKIVLRALRDLTRIWNAATTTAFLVCLVKMVRGQMVQGILWLLLITGVQGHLDCKPEYSYAIAKSDRIGLQGAENLTTVWKDYSHGMTLEDTMVIAWCKDGKLTYYARCTRETRYLAILHSRALPTSVVFKKLFEGQGQEDTVEMDDNFEFGLCPCDAKPIVRGTYNTTLLNGPAFQMVCPIGWTGTVSCMLANRDTLDTAVVRTYRRSRPFPYRQGCITQKTLGEDLYDCILGGNWTCVTGDQLQYTGGSVESCKWCGFKFQKNEGLPHYPIGKCRLKNETGYRFVDGTSCNREGVAIVPQGLVKCKIGDTIVQVIALDTKLGPMPCKPYEIIPSEGPVEKTACTFNYTRTLKNKYFEPRDSYFQQYMLKGEYQYWFDLEVTDHHRDYFAESILVVVVALLGGRYVLWLLVTYMVLSEQRASGTQYGAGEVVMMGNLLTHDSVEVVTYFLLLYLLLREENVKKWVLLLYHILVAHPLKSVTVILLMIGDVVKADPGDQGYMGQIDVCFTMIVLIVIGLIIARRDPTIVPLVTIVASLRVTGLTYGPGVDVAIAVMTTTLLMVTYVTDYFRYKRWLQCILSLVSGVFLIRSLIHIGRIEMPEVTIPNWRPLTLILLYLISTTVVTLWKVDLAGLLLQGVPILLMVATLWADFLTLILILPTYELVKLYYLKAVKTDIERSWLGGLSYKRVNSIYDVDESGEGVYLFPSKQKAQINYSILWPLVRATLISCVSSKWQLIYMAYLTVDFIYYMHRKVIEEISGGTNMISRIVAALIELNWSIEEEEIKGLKKFYLLSGRLRNLIIKHKVRNDTVARWYGEEEVYGMPKIMAIIRASTLSKNRHCIICTVCEGRNWKGGTCPKCGRHGIPIMCGTLLADFEERHYKRIFIREGNFDGPFRQEYNGFIQYTARGQLSLRNLPILATKVKMLMVGNLGEEVGDLEHLGWILRGPAVCKKITEHEKCHISILDKLTAFFGIMPRGTTPRAPVRFPTSLLKVRRGLETGWAYTHQGGISSVDHVTAGKDLLVCDSMGRTRVVCQSNNRLTDETEYGVKTDSGCPDGARCYVLNPEAVNVSGSKGAVVHLQKTGGEFTCVTASGTPAFFDLKNLKGWSGLPIFEASSGRVVGRVKVGKNEESKPTKIMSGIQTVSKSTADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPEVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVVDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNSRKLGEDVPVYIYATEDEDLAVDLLGLDWPDPGNQQVVETGKALKQVAGLSSAENALLVALFGYVGYQALSKRHVPMITDIYTIEDQRLEDTTHLQYAPNAIKTEGTETELKELASGDVEKIMGAISDYAAGGLEFVRSQAERIKTAPLFKENVEAAKGYVQKLINSLIEDKDVIIRYGLWGTHTALYKSIAARLGHETAFATLVLKWLAFGGESVSDHVRQAAVDLVVYYVMNKPSFPGDSETQQEGRRFVASLFISALTTYTYKTWNYHNLSKVVEPALAYLPYATSALKMFTPTRLESVVILSTTIYKTYLSIRKGKSDGLLGTGISAAMEILSQNPASVGISVMLGVGAIAAHNAIESSEQKRTLLMKVFVKNFLDQAATDELVKENPEKIIMALFEAVQTIGNPLRLIYHLYGVYYKGWEAKELSERTAGRNLFTLIMFEAFELLGMDSEGKIRNLSGNYILDLIYGLHKQINRRLKKMVLGWAPAPFSCDWTPGDERIRLPTDNYLRVETRCPCGYQMKAIKNVGGELTKVEERGPFLCRNRPGRGPVNYRVTKYYDDDLREIKPVAKLEGLVEHYYKGVTAKIDYSEGKTLLATDKWEVEHGTITRLAKRYTGVGFNGAYLGDQPNHRDLVERDCATITKNTVQFLKMKKGCAFTYDLTISNLTRLIELVHRNNLEEKEIPAATVTTWLAYTFVNEDVGTIRPVLGERVIPDPVVDVNLQPEVQVDTSEVGITIIGREALMTTGTTPVVEKVKPDADNNKNSVKVGLEEGNYPGPGVQTRTLVEEIHNRDTRPFIIVLGSKSSMSNRAKTARNINLYTGNDPREIRDLMAEGRMLVVALRDVDPDLHELVDFRGTFLDREALEALSLGQPKPKQVTKAAIRELLKEEDQVEIPDWFTSDDPVFLEVAMKTDKYHLVGDVGEVKNQAKALGATDQTRIVKEVGSRTYTMKLSSWFLQASNKQMSLTPLFEELLLRCPPATKSSKGHMASAYQLAQGNWEPIGCGVHLGTVPARRVKIHPYEAYLKLKSLLEEEEEKPRIKDTVMREHNKWILKKIRFQGNLRTKKILNPGKLSEQLDREGHKRNIYNNQISTIMSGAGIRLEKLPIVRAQTDTKSFHEAIRDKIDKDENRQNPELHNKLLEIFHTIAHPTLRHTYGEVTWEQLEAGINRKGAAGFLEKKNIGEVLDSEKHLVEQLIRDLKAGRKVKYYETAIPKNEKRDVSDDWQAGDLVGEKKPRVIQYPEAKIRLAITKVMYNWVKQQPVVIPGYEGKTPLFNIFNKVRKEWDLFNEPVAVSFDTKAWDTQVTSRDLQLIGEIQKYYYKKEWHKFIDTITDHMVEVPVITADGEVYIRNGQRGSGQPDTSAGNSMLNVLTMMYAFCESTGVPYKSFNRVARIHVCGDDGFLITEKGLGLKFANKGMQILHEAGKPQKITEGEKMKVAYRFEDIEFCSHTPVPVRWSDNTSSYMAGRDTAVILSKMATRLDSSGERGTTAYEKAVAFSFLLMYSWNPLVRRICLLVLSQRPEVAPSAQTTYYYKGDPIGAYKDVIGRNLSELKRTGFEKLANLNLSLSTLGIWTKHTSKRIIQDCVAIGKEEGNWLVNADRLISSKTGHLYIPDKGFTLQGRHYEQLQLGARTNPTTGVGTERYKLGPIVNLLLRRLKVLLMAAVGASG